(19) United States
(12) Patent Application Publication
(10) Pub. No.: US 2025/0380870 A1
(43) Pub. Date: Dec. 18, 2025

(54) FRACTIONAL FLOW RESERVE CALCULATION METHODS, SYSTEMS, AND STORAGE MEDIUMS

(71) Applicant: Canon U.S.A., Inc., Melville, NY (US)

(72) Inventor: Lampros Athanasiou, Medford, MA (US)

(21) Appl. No.: 19/141,003

(22) PCT Filed: Dec. 19, 2023
(86) PCT No.: PCT/US2023/084951

§ 371 Date: Jun. 18, 2025

Related U.S. Application Data
(60) Provisional application No. 63/476,607, filed on Dec. 21, 2022.

(51) Int. Cl. A61B 5/00; A61B 5/02; A61B 5/0215; A61B 5/026

(52) U.S. Cl. CPC A61B 5/0066; A61B 5/02007; A61B 5/02154; A61B 5/0261; A61B 5/7425

(57) ABSTRACT

One or more devices, systems, methods and storage mediums for optical imaging medical devices, such as, but not limited to, Optical Coherence Tomography (OCT), single mode OCT, and/or multi-modal OCT apparatuses and systems, and methods and storage mediums for use with same, for viewing, controlling, updating, and emphasizing one or more imaging modalities and/or for calculating one or more Fractional Flow Reserve (FFR) values or measurements are provided herein. Examples of applications include imaging, evaluating, and diagnosing biological objects, such as, but not limited to, for Gastro-intestinal, cardio, and/or ophthalmic applications, and being obtained via one or more optical instruments, such as, but not limited to, optical probes, catheters, and endoscopes. Techniques provided herein improve processing and imaging efficiency while achieving images that are more precise, and achieve imaging devices, systems, methods, and storage mediums that reduce mental and physical burden, that cost less, and that improve ease of use.

### CROSS-REFERENCE TO RELATED APPLICATION(S)

This application relates, and claims priority, to U.S. Prov. Patent Application Ser. No. 63/476,607, filed Dec. 21, 2022, the disclosure of which is incorporated by reference herein in its entirety.

### FIELD OF THE INVENTION

This present disclosure generally relates to computer imaging and/or to the field of optical imaging, particularly to devices/apparatuses, systems, methods, and storage mediums for calculating Fractional Flow Reserve (FFR) values or measurements and/or for using one or more imaging modalities, such as, but not limited to, angiography, Optical Coherence Tomography (OCT), Multi-modality OCT (MM-OCT), near-infrared fluorescence (NIRF), near-infrared auto-fluorescence (NIRAF), OCT-NIRF, OCT-NIRAF, robot imaging, snake robot imaging, etc. Examples of OCT applications include imaging, evaluating, and diagnosing biological objects, such as, but not limited to, for gastro-intestinal, pulmonary, cardio, ophthalmic and/or intravascular applications, and being obtained via one or more optical instruments, such as, but not limited to, one or more optical probes, one or more catheters, one or more endoscopes, one or more capsules (e.g., one or more tethered capsules), and one or more needles (e.g., a biopsy needle). One or more devices, systems, methods and storage mediums for characterizing, examining and/or diagnosing, and/or measuring a target, sample, or object in application(s) using an apparatus or system that uses and/or controls one or more imaging modalities are discussed herein.

### BACKGROUND OF THE INVENTION

Fiber optic catheters and endoscopes have been developed to access to internal organs. For example in cardiology, Optical Coherence Tomography (OCT) has been developed to see depth resolved images of vessels with a catheter. The catheter, which may include a sheath, a coil and an optical probe, may be navigated to a coronary artery.

OCT is a technique for obtaining high-resolution cross-sectional images of tissues or materials, and enables real time visualization. The aim of the OCT techniques is to measure the time delay of light by using an interference optical system or interferometry, such as via Fourier Transform or Michelson interferometers. A light from a light source delivers and splits into a reference arm and a sample (or measurement) arm with a splitter (e.g., a beamsplitter). A reference beam is reflected from a reference mirror (partially reflecting or other reflecting element) in the reference arm while a sample beam is reflected or scattered from a sample in the sample arm. Both beams combine (or are recombined) at the splitter and generate interference patterns. The output of the interferometer is detected with one or more detectors, such as, but not limited to, photodiodes or multi-array cameras, in one or more devices, such as, but not limited to, a spectrometer (e.g., a Fourier Transform infrared spectrometer). The interference patterns are generated when the path length of the sample arm matches that of the reference arm to within the coherence length of the light source. By evaluating the output beam, a spectrum of an input radiation may be derived as a function of frequency. The frequency of the interference patterns corresponds to the distance between the sample arm and the reference arm. The higher frequencies are, the more the path length differences are. Single mode fibers may be used for OCT optical probes, and double clad fibers may be used for fluorescence and/or spectroscopy.

A multi-modality system such as an OCT, fluorescence, and/or spectroscopy system with an optical probe is developed to obtain multiple information at the same time. During vascular diagnosis and intervention procedures, such as Percutaneous Coronary Intervention (PCI), users of optical coherence tomography (OCT) sometimes have difficulty understanding the tomography image in correlation with other modalities because of an overload of information, which causes confusion in image interpretation. PCI, and other vascular diagnosis and intervention procedures, have improved with the introduction of intravascular imaging (IVI) modalities, such as, but not limited to, intravascular ultrasound (IVUS) and optical coherence tomography (OCT).

Coronary blood flow plays an important role in oxygenizing the heart and reducing the risk of an adverse coronary artery disease (CAD) outcome. Reduced blood flow due to stenosis can cause an ischemic heart disease. Physiological assessment of coronary artery disease, such as fractional flow reserve (FFR) and instantaneous wave-free ratio (iFR), is one of the important tools to decide whether patients should undergo percutaneous coronary intervention (PCI) and/or to evaluate the procedural success of PCI. Evaluation of the ischemic burden of coronary stenosis plays a role in successful outcomes for PCI procedure(s).

Although angiography is used as an imaging method during PCI, angiography has a substantial mismatch between stenosis severity and ischemia [2]-[4], [5]. Also, the spatial resolution of angiography (0.2 mm) is not desirable. As such, FFR tends to be used for PCI evaluations instead of angiography [5].

FFR is a current way of evaluating the ischemic burden and requires the use of a specialized pressure catheter. A study has shown that using FFR-guided PCI demonstrated a 30% decrease in adverse PCI outcomes within the first post-PCI year [1]. Virtual FFR methods may be applied for PCI procedure using multiple catheters. However, most virtual FFR methods either cannot be applied real-time or have limited agreement with catheter-based FFR measurements. Additionally, virtual FFR methods may have limitations relating to the presence of arterial branches, which can distribute the blood flow and lead to variation in virtual FFR values and relating to the low spatial resolution of angiography [13].

Since IVI resolution (for example, OCT has 0.02 mm resolution) is superior to the angiography resolution (0.2 mm) [13], several types of IVI-derived FFR have been developed [14]-[19]. Among the OCT-based FFR methods, Optical Flow Ratio (OFR) has been approved (CE mark) for clinical use. In the OFR method, a hyperemic flow rate is calculated by multiplying a fixed flow velocity of 0.35 m/s by a patient-specific reference lumen and applied to an algorithm which computes the FFR. However, the use of a fixed flow velocity imposes certain issues since one of the main characteristics of coronary circulation is the change of coronary flow velocity according to the coronary stenosis [13], [20]. Moreover, wire based FFR is evaluated under hyperemia (maximum coronary flow under maximum exercise or drugs).

Although several virtual FFR methods were developed, measurements coming from a specialized pressure catheter, used in parallel with an intravascular imaging catheter, are still considered as the better option. However, virtual FFR methods have two major drawbacks: (i) either the virtual FFR methods do not calculate patient specific values; or (ii) the virtual FFR methods do not take into account the pressure loss due to the presence of arterial breaches. Such issues increase cost and increase the interventional risk during PCI procedure(s).

Accordingly, it would be desirable to provide at least one imaging or optical apparatus/device, system, method, and storage medium that may use one or more imaging modalities and that may use one or more FFR calculation processes or techniques that operate to reduce both the cost and the interventional risk during PCI procedure(s).

### SUMMARY OF THE INVENTION

Accordingly, it is a broad object of the present disclosure to provide imaging (e.g., OCT, IVI, IVUS, NIRF, NIRAF, SNAKE robots, robots, etc.) apparatuses, systems, methods and storage mediums for using and/or controlling multiple imaging modalities and/or for fractional flow reserve calculation technique(s)/process(es). It is also a broad object of the present disclosure to provide OCT devices, systems, methods and storage mediums using an interference optical system, such as an interferometer (e.g., spectral-domain OCT (SD-OCT), swept-source OCT (SS-OCT), multimodal OCT (MM-OCT), Intravascular Ultrasound (IVUS), Near-Infrared Autofluorescence (NIRAF), Near-Infrared Spectroscopy (NIRS), Near-Infrared Fluorescence (NIRF), therapy modality using light, sound, or other source of radiation, etc.), that may use FFR calculation technique(s) discussed herein.

One or more embodiments of the present disclosure provides FFR techniques that may be used to reduce both the cost and the interventional risk during PCI procedures.

One or more embodiments of the present disclosure may calculate a virtual FFR using intravascular imaging (e.g., such as, but not limited to, OCT imaging, IVUS imaging, another imaging modality imaging, etc.). In one or more embodiments, a Coronary flow reserve (CFR) may be adjusted according to or based on the coronary stenosis severity, and one or more embodiments may simultaneously take into account a branch flow distribution by adjusting a pressure difference between the stenosis accordingly.

One or more aspects of the FFR calculation technique(s) of the present disclosure were evaluated using several validation metrics and demonstrated that a use of arterial branch adjustment(s) of the present disclosure may improve the FFR accuracy in comparison to a CRF stenotic-adjustment technique. As such, one or more features of the present disclosure improve the applicability of virtual OCT-FFR technique(s) in one or more clinical settings.

In one or more embodiments, a real-time intravascular imaging based virtual FFR method(s) or technique(s) may be employed that account for arterial branch flow distribution, and may increase the level of agreement with the catheter-based FFR method(s).

One or more embodiments of the present disclosure may use virtual FFR methods to optimize PCI procedure, reduce cost, reduce time, and reduce risk, including embodiments using one or multiple catheters. FFR values may vary from one embodiment to the next. In one or more embodiments, FFR values from 0.8-1.0 indicate no myocardial ischemia, while an FFR value lower than 0.75-0.80 indicates an association with myocardial ischemia (indication for PCI). FFR may be measured during routine coronary angiography by using a pressure catheter to calculate the ratio between coronary pressure distal to a coronary artery stenosis, and aortic pressure under conditions of maximum myocardial hyperemia. The ratio may represent the potential decrease in coronary flow distal to the coronary stenosis in one or more embodiments. One or more embodiments may combine the variation flow velocity and hyperemia conditions such that patient specific virtual FFR values may be calculated more efficiently or accurately (as compared to situations where the variation flow velocity and hyperemia conditions are not combined or used in an embodiment).

One or more embodiments of the present disclosure may calculate the FFR and provide information on treatment option(s) for the treatment of stenosis and/or another medical condition. One or more methods of the present disclosure may calculate FFR and may automatically decide or a user may decide to treat or not treat stenosis and/or other condition(s). One or more methods of the present disclosure may use FFR in real-time. One or more embodiments of the present disclosure may include an OCT FFR method that uses anatomic information (e.g., a volume of a vessel, any other anatomic information discussed in the present disclosure, etc.); etc.), to plan PCI during a procedure, and to assess procedural success of the PCI more accurately.

One or more embodiments of the present disclosure may achieve or operate to do one or more of the following: (i) fully and automatically calculate an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculate one or more arterial branches; (iii) calculate (e.g., manually or automatically) an arterial pressure loss due to the arterial branches; and/or (iv) derive one or more patient specific FFR measurements.

One or more embodiments of an image processing apparatus of the present disclosure may include: one or more processors that operate to one or more of the following: (i) fully and automatically calculate an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculate one or more arterial branches; (iii) calculate (e.g., manually or automatically) an arterial pressure loss due to the arterial branches; and/or (iv) derive one or more patient specific FFR measurements.

One or more methods or storage mediums of the present disclosure may achieve or operate to one or more of the following: (i) fully and automatically calculate an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculate one or more arterial branches; (iii) calculate (e.g., manually or automatically) an arterial pressure loss due to the arterial branches; and/or (iv) derive one or more patient specific FFR measurements.

One or more embodiments of the present disclosure may automatically calculate FFR using one or more area stenosis calculation algorithms or methods of the present disclosure. For example, in one or more embodiments, images of only one imaging modality (e.g., OCT only, IVUS only, any other imaging modality discussed herein only, etc.) may be used to calculate FFR automatically.

One or more embodiments of the present disclosure may automatically detect one or more arterial branches and may calculate an arterial pressure loss using, in, or during one or more FFR calculations.

One or more embodiments of the present disclosure may detect an arterial FFR using imaging data: (i) by detecting a stenotic area automatically, the FFR measurement(s) may be calculated for the stenotic area only, where the pressure (e.g., a blood pressure, an arterial pressure, a structural pressure, etc.) may be changing; (ii) by using the branch detection method(s) of the present disclosure, the arterial branch(es) may be accurately detected; and/or (iii) by using the branch detection method(s) of the present disclosure, the FFR values may be calculate more accurately (as compared to a situation where the branch detection method(s) of the present disclosure are not being used).

One or more embodiments of the present disclosure may use one or more of the following: (i) automatic, minimum lumen and normal area extraction; (ii) calculation of an imaging modality-derived pressure loss (e.g., OCT-derived pressure loss, IVUS-derived pressure loss, other imaging modality-derived pressure loss, etc.); (iii) arterial branch FFR adjustment(s); (iv) calculation of a stenotic flow reserve (SFR); and/or (v) determining and/or processing results of one or more methods or techniques discussed herein.

In one or more embodiments, the one or more processors may further operate to one or more of the following: (i) obtain intravascular image data (e.g., for a pullback); (ii) detect lumen area(s) using a lumen detection method or technique; (iii) detect a minimum lumen area (As) and define a stenotic area (L); (iv) construct a carpet view (e.g., of the pullback) and automatically calculate the area(s) of any arterial branch(es); (v) in a case where an arterial branch is within (or has a portion that passes through or is within) the stenotic area, reduce a velocity of a fluid (e.g., blood) or object passing through the branch or lumen; (vi) calculate a diastolic and systolic (e.g., of a patient, of a specific patient, for one or more patients, for an object or sample, etc.) Stenotic Flow Reserve(s) (SFR) using the velocity, the stenotic area (L), and the minimum lumen area (As); and/or (vii) using the SFR, calculate the Fractional Flow Reserve (FFR).

In one or more embodiments, the object may be a blood vessel or artery, and the acquisition location may be a region that is diseased, may be a region that a physician(s), clinician(s) or other user(s) of the apparatus is/are considering for further assessment, and/or may be a region of an object or sample being evaluated. In one or more embodiments, one or more processors may operate to calculate FFR.

In one or more embodiments, one or more processors may further operate to one or more of the following: (i) display an image for each of one or more imaging modalities on a display, wherein the one or more imaging modalities include one or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; (ii) display an image for each of one or more imaging modalities on a display, wherein the one or more imaging modalities include one or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; and (iii) change or update the displays for each of the one or more imaging modalities based on a calculated FFR and/or based on a request to update or change the displays after calculating the FFR.

In one or more embodiments, one or more processors may further operate to one or more of the following: (i) receive information for an interventional device to be used for a Percutaneous Coronary Intervention (PCI); and (ii) in a case where the interventional device is a stent, perform one or more of: detecting stent expansion or underexpansion, detecting stent apposition or malapposition, performing co-registration, performing imaging, displaying a notification regarding the detected stent expansion or underexpansion, displaying a notification regarding the detected stent apposition or malapposition, and confirming stent placement.

In one or more embodiments, one or more processors may employ computational fluid dynamics (CFD). For example, one or more embodiments of the present disclosure may employ information on two-dimensional (2D) or three-dimensional (3D) results and/or structure(s) for the object in order to construct a CFD model for the object.

In one or more embodiments of the present disclosure, at least one method for calculating or deriving FFR measurements (and at least one storage medium having one or more programs stored therein that operate to cause a computer or processor to perform a method(s), where the method) may include: (i) fully and automatically calculating an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculating one or more arterial branches; (iii) calculating (e.g., manually or automatically) an arterial pressure loss due to the arterial branches; and/or (iv) deriving one or more patient specific FFR measurements.

In one or more embodiments of the present disclosure, at least one method for calculating or deriving FFR measurements (and at least one storage medium having one or more programs stored therein that operate to cause a computer or processor to perform a method(s), where the method) may include: (i) obtaining intravascular image data (e.g., for a pullback); (ii) detecting lumen area(s) using a lumen detection method or technique; (iii) detecting a minimum lumen area (As) and define a stenotic area (L); (iv) constructing a carpet view (e.g., of the pullback) and automatically calculating the area(s) of any arterial branch(es); (v) in a case where an arterial branch is within (or has a portion that passes through or is within) the stenotic area, reducing a velocity of a fluid (e.g., blood) or object passing through the branch or lumen; (vi) calculating a diastolic and systolic (e.g., of a patient, of a specific patient, for one or more patients, for an object or sample, etc.) Stenotic Flow Reserve(s) (SFR) using the velocity, the stenotic area (L), and the minimum lumen area (As); and/or (vii) using the SFR, calculating the Fractional Flow Reserve (FFR).

In one or more embodiments, an apparatus may include one or more processors that operate to: obtain one or more intravascular images of one or more imaging modalities of an object or target during a pullback of a probe or catheter; calculate or determine a pressure loss or change of one or more arterial branches detected in the one or more intravascular images; and automatically calculate one or more Fractional Flow Reserve (FFR) values using the one or more intravascular images and using the calculated or determined pressure loss or change of the one or more arterial branches. The one or more processors may further operate to one or more of the following: detect lumen area(s) using a lumen detection method or technique; detect a minimum lumen area (As) and define a stenotic area (L); construct a carpet view of the pullback and automatically calculate the area(s) of the detected one or more arterial branches; in a case where an arterial branch is within or has a portion that passes through or is within the stenotic area, reduce a velocity of a fluid or other object passing through the branch or lumen; calculate a diastolic and systolic Stenotic Flow Reserve(s) (SFR) using the velocity, the stenotic area (L), and the minimum lumen area (As); and/or use the SFR to calculate the Fractional Flow Reserve (FFR). The one or more processors may further operate to detect the one or more arterial branches in the one or more intravascular images. The one or more processors may further operate to detect a stenotic area in the one or more intravascular images and to calculate the FFR values for the stenotic area only where the pressure loss or change of the one or more arterial branches is occurring. In one or more embodiments, the object or target is an organ, a tissue, a sample, a portion of a patient, a vessel, a blood vessel, or a patient.

In one or more embodiments, the one or more processors may further operate to: determine whether a Percutaneous Coronary Intervention (PCI) is needed for the object or target; in a case where it is determined that the object or target needs the PCI, perform the PCI, or, in a case where it is determined that the object or target does not need the PCI, save the images; in a case where the PCI is to be performed, plan the PCI; in a case where the PCI is performed, assess or evaluate procedural success of the PCI; evaluate the physiology of the object or target; and/or in a case where the object is a vessel or blood vessel, evaluate the physiology of the vessel and/or a lesion of the vessel. The one or more processors may further operate to reduce a cost of using the image processing apparatus and to reduce an interventional risk during PCI procedure(s).

The one or more processors may further operate to one or more of the following: (i) display an image for each of the one or more imaging modalities on a display, wherein the one or more imaging modalities include one or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; (ii) display an image for each of the one or more imaging modalities on a display, wherein the one or more imaging modalities include two or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; and (iii) change or update the displays for each of the one or more imaging modalities based on a calculated FFR and/or based on a request to update or change the displays after calculating the FFR. The one or more processors may further operate to one or more of the following: (i) receive information for an interventional device to be used for a Percutaneous Coronary Intervention (PCI); and/or (ii) in a case where the interventional device is a stent, perform one or more of: detecting stent expansion or underexpansion, detecting stent apposition or malapposition, performing co-registration, performing imaging, displaying a notification regarding the detected stent expansion or underexpansion, displaying a notification regarding the detected stent apposition or malapposition, and confirming stent placement.

In one or more embodiments, the one or more processors may operate to one or more of the following: (i) employ information on a two-dimensional (2D) and/or three-dimensional (3D) structure or structures for the object to create or construct/reconstruct a computational fluid dynamics (CFD) model or result for the object; (ii) use 2D or 3D results and/or 2D or 3D structure(s) and calculate the one or more FFR values and/or one or more instantaneous wave-free ratio (iFR) values; (iii) employ computational fluid dynamics (CFD) to calculate one or more pressures and to have or obtain the one or more FFR values and/or one or more instantaneous wave-free ratio (iFR) values; (iv) calculate the one or more FFR values and providing information on treatment option(s) for the treatment of stenosis and/or another medical condition; (v) use the one or more FFR values and/or one or more instantaneous wave-free ratio (iFR) values in real-time; (vi) calculate pressure(s) and/or include a lamp parameter/circuit analog model; (vii) include or use an Optical Coherence Tomography (OCT) or Intravascular Ultrasound (IVUS) images or frames FFR method that uses anatomic information; and/or (viii) process anatomic information where the anatomic information includes at least a volume of a vessel.

In one or more embodiments, a method for calculating Fractional Flow Reserve (FFR) values may include: obtaining one or more intravascular images of one or more imaging modalities of an object or target during a pullback of a probe or catheter; calculating or determining a pressure loss or change of one or more arterial branches detected in the one or more intravascular images; and automatically calculating one or more Fractional Flow Reserve (FFR) values using the one or more intravascular images and using the calculated or determined pressure loss or change of the one or more arterial branches. The method(s) may further comprise one or more of the following: detecting lumen area(s) using a lumen detection method or technique; detecting a minimum lumen area (As) and defining a stenotic area (L); constructing a carpet view of the pullback and automatically calculating the area(s) of the detected one or more arterial branches; in a case where an arterial branch is within or has a portion that passes through or is within the stenotic area, reducing a velocity of a fluid or other object passing through the branch or lumen; calculating a diastolic and systolic Stenotic Flow Reserve(s) (SFR) using the velocity, the stenotic area (L), and the minimum lumen area (As); and/or using the SFR to calculate the Fractional Flow Reserve (FFR). The method(s) may further include detecting the one or more arterial branches in the one or more intravascular images. The method(s) may further include detecting a stenotic area in the one or more intravascular images and calculating the FFR values for the stenotic area only where the pressure loss or change of the one or more arterial branches is occurring. In one or more methods, the object or target may be an organ, a tissue, a sample, a portion of a patient, a vessel, a blood vessel, or a patient.

In one or more embodiments, the methods may include one or more of the following: determining whether a Percutaneous Coronary Intervention (PCI) is needed for the object or target; in a case where it is determined that the object or target needs the PCI, performing the PCI, or, in a case where it is determined that the object or target does not need the PCI, saving the images in a memory; in a case where the PCI is to be performed, planning the PCI; in a case where the PCI is performed, assessing or evaluating procedural success of the PCI; evaluating the physiology of the object or target; and/or in a case where the object is a vessel or blood vessel, evaluating the physiology of the vessel and/or a lesion of the vessel. The method(s) may further include reducing a cost of calculating the one or more FFR values as compared to a case not using the method, and reducing an interventional risk during PCI procedure(s).

The method(s) may further include one or more of the following: (i) displaying an image for each of the one or more imaging modalities on a display, wherein the one or more imaging modalities include one or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; (ii) displaying an image for each of the one or more imaging modalities on a display, wherein the one or more imaging modalities include two or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; and (iii) changing or updating the displays for each of the one or more imaging modalities based on a calculated FFR and/or based on a request to update or change the displays after calculating the FFR. The method(s) may further include one or more of the following: (i) receiving information for an interventional device to be used for a Percutaneous Coronary Intervention (PCI); and/or (ii) in a case where the interventional device is a stent, performing one or more of: detecting stent expansion or underexpansion, detecting stent apposition or malapposition, performing co-registration, performing imaging, displaying a notification regarding the detected stent expansion or underexpansion, displaying a notification regarding the detected stent apposition or malapposition, and confirming stent placement.

The method(s) may further include one or more of the following: (i) employing information on a two-dimensional (2D) and/or three-dimensional (3D) structure or structures for the object to create or construct/reconstruct a computational fluid dynamics (CFD) model or result for the object; (ii) using 2D or 3D results and/or 2D or 3D structure(s) and calculating the one or more FFR values and/or one or more instantaneous wave-free ratio (iFR) values; (iii) employing computational fluid dynamics (CFD) to calculate one or more pressures and to have or obtain the one or more FFR values and/or one or more instantaneous wave-free ratio (iFR) values; (iv) calculating the one or more FFR values and providing information on treatment option(s) for the treatment of stenosis and/or another medical condition; (v) using the one or more FFR values and/or one or more instantaneous wave-free ratio (iFR) values in real-time; (vi) calculating pressure(s) and/or include a lamp parameter/circuit analog model; (vii) including or using an Optical Coherence Tomography (OCT) or Intravascular Ultrasound (IVUS) images or frames FFR method that uses anatomic information; and/or (viii) processing anatomic information where the anatomic information includes at least a volume of a vessel.

In one or more embodiments, a non-transitory computer-readable storage medium storing at least one program for causing a computer to execute a method for calculating one or more Fractional Flow Reserve (FFR) values, the method may include: obtaining one or more intravascular images of one or more imaging modalities of an object or target during a pullback of a probe or catheter; calculating or determining a pressure loss or change of one or more arterial branches detected in the one or more intravascular images; and automatically calculating one or more Fractional Flow Reserve (FFR) values using the one or more intravascular images and using the calculated or determined pressure loss or change of the one or more arterial branches. In one or more storage medium embodiments, the method may further include one or more of the following: detecting lumen area(s) using a lumen detection method or technique; detecting a minimum lumen area (As) and defining a stenotic area (L); constructing a carpet view of the pullback and automatically calculating the area(s) of the detected one or more arterial branches; in a case where an arterial branch is within or has a portion that passes through or is within the stenotic area, reducing a velocity of a fluid or other object passing through the branch or lumen; calculating a diastolic and systolic Stenotic Flow Reserve(s) (SFR) using the velocity, the stenotic area (L), and the minimum lumen area (As); and/or using the SFR to calculate the Fractional Flow Reserve (FFR).

The present disclosure describes a means to allow OCT users to focus on the area of interest in one or more imaging modalities, such as, but not limited to, the aforementioned imaging modalities, any other imaging modalities discussed herein, etc.

As described herein, one or more embodiments of the present disclosure may provide at least one imaging or optical apparatus/device, system, method, and storage medium that may use one or more imaging modalities and that may use one or more FFR calculation processes or techniques that operate to reduce both the cost and the interventional risk during PCI procedure(s).

When the user obtains an intravascular image at a location within the object, that specific portion of the object may be at a predetermined location based on prior angiographic images or other information.

While more than one angiography image or intravascular image may be used in one or more embodiments of the present disclosure, at least one intravascular image may be used in one or more embodiments.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods and one or more storage mediums using OCT and/or other imaging modality technique(s) to calculate or process FFR are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1A is a schematic diagram showing at least one embodiment of a system that may be used for performing one or multiple imaging modality viewing and control, and one or more other methods, in accordance with one or more aspects of the present disclosure;

FIG. 1B is a schematic diagram illustrating an imaging system for executing one or more steps to process image data and/or to use one or more methods in accordance with one or more aspects of the present disclosure;

FIG. 2 is a flowchart of at least one embodiment of a method or procedure that may be used in accordance with one or more aspects of the present disclosure;

FIG. 3 is a diagram of at least one embodiment of a catheter that may be used with one or more embodiments for FFR method(s) or algorithm(s) in accordance with one or more aspects of the present disclosure;

FIG. 4 is at least one graphic representation of at least one embodiment of a stenotic frame and a stenosis area in a pullback that may be used with, or obtained from, one or more method(s) or algorithm(s) in accordance with one or more aspects of the present disclosure;

FIGS. 5A-5C show at least one embodiment of a carpet view, a detected branch area, and another detected branch area, respectively, that may be viewed or obtained using one or more methods in accordance with one or more aspects of the present disclosure;

FIGS. 6A-6B show graphs for at least one embodiment of a calculation and concept of stenotic flow reserve (SFR) on diastolic (FIG. 6A) and systolic (FIG. 6B) phase(s) that may be used in accordance with one or more aspects of the present disclosure;

FIG. 7 shows a graph for at least one embodiment of the method(s) of the present disclosure versus a wire based FFR in accordance with one or more aspects of the present disclosure;

FIG. 8A shows at least one embodiment of an OCT apparatus or system for utilizing one or more imaging modalities and/or one or more FFR methods in accordance with one or more aspects of the present disclosure;

FIG. 8B shows at least another embodiment of an OCT apparatus or system for utilizing one or more imaging modalities and/or one or more FFR methods in accordance with one or more aspects of the present disclosure;

FIG. 8C shows at least a further embodiment of an OCT and NIRAF apparatus or system for utilizing one or more imaging modalities and/or one or more FFR methods in accordance with one or more aspects of the present disclosure;

FIG. 9 is a flow diagram showing a method of performing an imaging feature, function or technique in accordance with one or more aspects of the present disclosure;

FIG. 10 shows a schematic diagram of an embodiment of a computer that may be used with one or more embodiments of an apparatus or system or one or more methods discussed herein in accordance with one or more aspects of the present disclosure; and

FIG. 11 shows a schematic diagram of another embodiment of a computer that may be used with one or more embodiments of an imaging apparatus or system or methods discussed herein in accordance with one or more aspects of the present disclosure.

### DETAILED DESCRIPTION OF THE PRESENT INVENTION

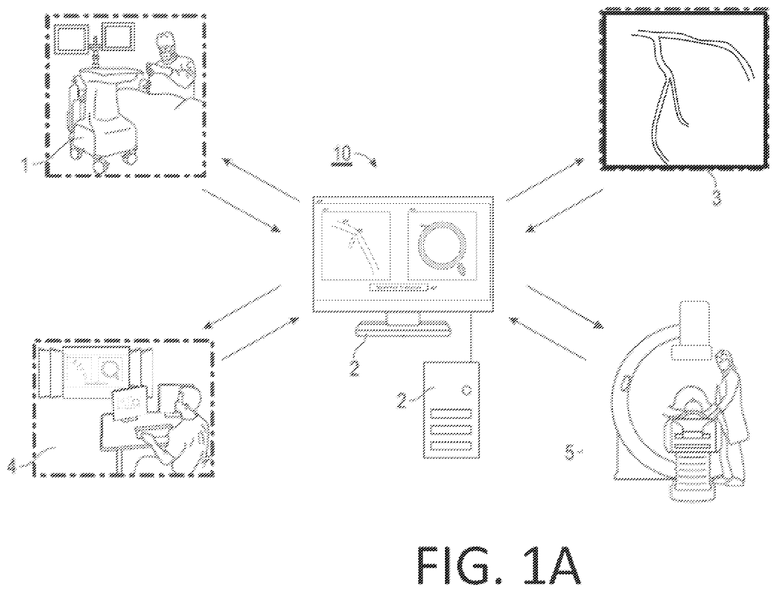

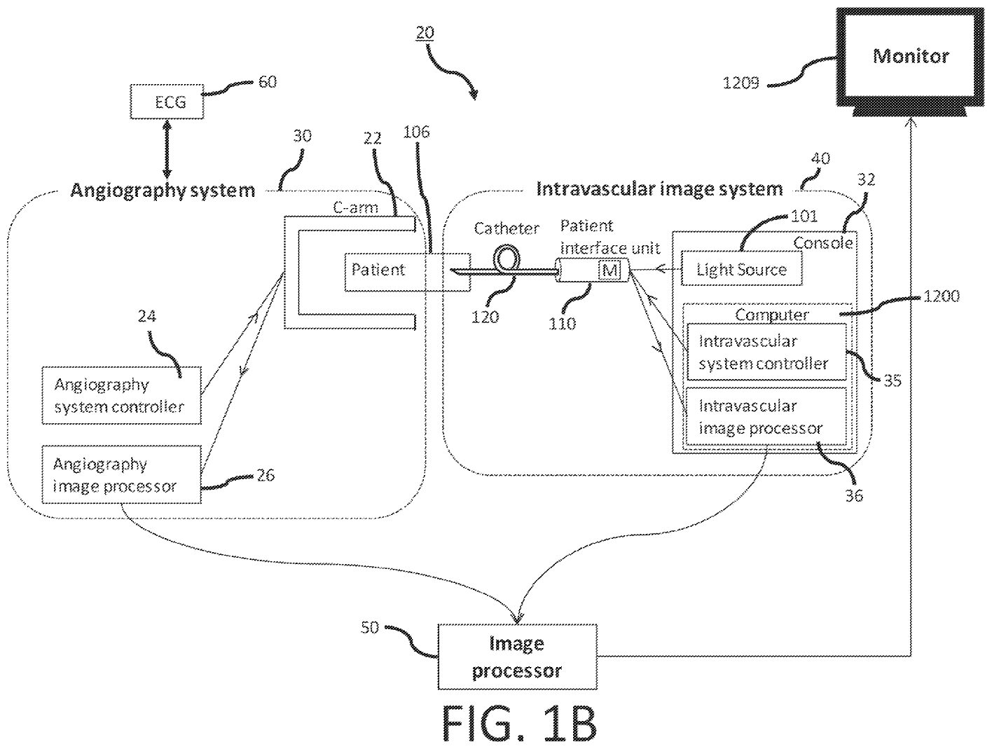

One or more devices, systems, methods and storage mediums for characterizing tissue, or an object, using one or more imaging techniques or modalities (such as, but not limited to, OCT, IVUS, fluorescence, NIRF, NIRAF, etc.) are disclosed herein. Several embodiments of the present disclosure, which may be carried out by the one or more embodiments of an apparatus, system, method and/or computer-readable storage medium of the present disclosure are described diagrammatically and visually in FIGS. 1A through 1I.

It is a broad object of the present disclosure to provide imaging (e.g., OCT, IVI, IVUS, NIRF, NIRAF, SNAKE robots, robots, etc.) apparatuses, systems, methods and storage mediums for using and/or controlling multiple imaging modalities and/or for fractional flow reserve calculation technique(s)/process(es). It is also a broad object of the present disclosure to provide OCT devices, systems, methods and storage mediums using an interference optical system, such as an interferometer (e.g., spectral-domain OCT (SD-OCT), swept-source OCT (SS-OCT), multimodal OCT (MM-OCT), Intravascular Ultrasound (IVUS), Near-Infrared Autofluorescence (NIRAF), Near-Infrared Spectroscopy (NIRS), Near-Infrared Fluorescence (NIRF), therapy modality using light, sound, or other source of radiation, etc.), that may use FFR calculation technique(s) discussed herein.

One or more embodiments of the present disclosure provides FFR techniques that may be used to reduce both the cost and the interventional risk during PCI procedures.

One or more embodiments of the present disclosure may calculate a virtual FFR using intravascular imaging (e.g., such as, but not limited to, OCT imaging, IVUS imaging, another imaging modality imaging, etc.). In one or more embodiments, a Coronary flow reserve (CFR) may be adjusted according to or based on the coronary stenosis severity, and one or more embodiments may simultaneously take into account a branch flow distribution by adjusting a pressure difference between the stenosis accordingly.

One or more aspects of the FFR calculation technique(s) of the present disclosure were evaluated using several validation metrics and demonstrated that a use of arterial branch adjustment(s) of the present disclosure may improve the FFR accuracy in comparison to a CRF stenotic-adjustment technique. As such, one or more features of the present disclosure improve the applicability of virtual OCT-FFR technique(s) in one or more clinical settings.

In one or more embodiments, a real-time intravascular imaging based virtual FFR method(s) or technique(s) may be employed that account for arterial branch flow distribution, and may increase the level of agreement with the catheter-based FFR method(s).

One or more embodiments of the present disclosure may use virtual FFR methods to optimize PCI procedure, reduce cost, reduce time, and reduce risk, including embodiments using one or multiple catheters. FFR values may vary from one embodiment to the next. In one or more embodiments, FFR values from 0.8-1.0 indicate no myocardial ischemia, while an FFR value lower than 0.75-0.80 indicates an association with myocardial ischemia (indication for PCI). FFR may be measured during routine coronary angiography by using a pressure catheter to calculate the ratio between coronary pressure distal to a coronary artery stenosis, and aortic pressure under conditions of maximum myocardial hyperemia. The ratio may represent the potential decrease in coronary flow distal to the coronary stenosis in one or more embodiments. One or more embodiments may combine the variation flow velocity and hyperemia conditions such that patient specific virtual FFR values may be calculated more efficiently or accurately (as compared to situations where the variation flow velocity and hyperemia conditions are not combined or used in an embodiment).

One or more embodiments of the present disclosure may calculate the FFR and provide information on treatment option(s) for the treatment of stenosis and/or another medical condition. One or more methods of the present disclosure may calculate FFR and may automatically decide or a user may decide to treat or not treat stenosis and/or other condition(s). One or more methods of the present disclosure may use FFR in real-time. One or more embodiments of the present disclosure may include an OCT FFR method that uses anatomic information (e.g., a volume of a vessel, any other anatomic information discussed in the present disclosure, etc.); etc.), to plan PCI during a procedure, and to assess procedural success of the PCI more accurately.

One or more embodiments of the present disclosure may achieve or operate to do one or more of the following: (i) fully and automatically calculate an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculate one or more arterial branches; (iii) calculate (e.g., manually or automatically) an arterial pressure loss due to the arterial branches; and/or (iv) derive one or more patient specific FFR measurements.

One or more embodiments of an image processing apparatus of the present disclosure may include: one or more processors that operate to one or more of the following: (i) fully and automatically calculate an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculate one or more arterial branches; (iii) calculate (e.g., manually or automatically) an arterial pressure loss due to the arterial branches; and/or (iv) derive one or more patient specific FFR measurements.

One or more methods or storage mediums of the present disclosure may achieve or operate to one or more of the following: (i) fully and automatically calculate an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculate one or more arterial branches; (iii) calculate (e.g., manually or automatically) an arterial pressure loss due to the arterial branches; and/or (iv) derive one or more patient specific FFR measurements.

One or more embodiments of the present disclosure may automatically calculate FFR using one or more area stenosis calculation algorithms or methods of the present disclosure. For example, in one or more embodiments, images of only one imaging modality (e.g., OCT only, IVUS only, any other imaging modality discussed herein only, etc.) may be used to calculate FFR automatically.

One or more embodiments of the present disclosure may automatically detect one or more arterial branches and may calculate an arterial pressure loss using, in, or during one or more FFR calculations.

One or more embodiments of the present disclosure may detect an arterial FFR using imaging data: (i) by detecting a stenotic area automatically, the FFR measurement(s) may be calculated for the stenotic area only, where the pressure (e.g., a blood pressure, an arterial pressure, a structural pressure, etc.) may be changing; (ii) by using the branch detection method(s) of the present disclosure, the arterial branch(es) may be accurately detected; and/or (iii) by using the branch detection method(s) of the present disclosure, the FFR values may be calculate more accurately (as compared to a situation where the branch detection method(s) of the present disclosure are not being used).

One or more embodiments of the present disclosure may use one or more of the following: (i) automatic, minimum lumen and normal area extraction; (ii) calculation of an imaging modality-derived pressure loss (e.g., OCT-derived pressure loss, IVUS-derived pressure loss, other imaging modality-derived pressure loss, etc.); (iii) arterial branch FFR adjustment(s); (iv) calculation of a stenotic flow reserve (SFR); and/or (v) determining and/or processing results of one or more methods or techniques discussed herein.

In one or more embodiments, the one or more processors may further operate to one or more of the following: (i) obtain intravascular image data (e.g., for a pullback); (ii) detect lumen area(s) using a lumen detection method or technique; (iii) detect a minimum lumen area (As) and define a stenotic area (L); (iv) construct a carpet view (e.g., of the pullback) and automatically calculate the area(s) of any arterial branch(es); (v) in a case where an arterial branch is within (or has a portion that passes through or is within) the stenotic area, reduce a velocity of a fluid (e.g., blood) or object passing through the branch or lumen; (vi) calculate a diastolic and systolic (e.g., of a patient, of a specific patient, for one or more patients, for an object or sample, etc.) Stenotic Flow Reserve(s) (SFR) using the velocity, the stenotic area (L), and the minimum lumen area (As); and/or (vii) using the SFR, calculate the Fractional Flow Reserve (FFR).

In one or more embodiments, the object may be a blood vessel or artery, and the acquisition location may be a region that is diseased, may be a region that a physician(s), clinician(s) or other user(s) of the apparatus is/are considering for further assessment, and/or may be a region of an object or sample being evaluated. In one or more embodiments, one or more processors may operate to calculate FFR.

In one or more embodiments, one or more processors may further operate to one or more of the following: (i) display an image for each of one or more imaging modalities on a display, wherein the one or more imaging modalities include one or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; (ii) display an image for each of multiple imaging modalities on a display, wherein the multiple imaging modalities include three or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; and (iii) change or update the displays for each of the multiple imaging modalities based on a calculated FFR and/or based on a request to update or change the displays after calculating the FFR.

In one or more embodiments, one or more processors may further operate to one or more of the following: (i) receive information for an interventional device to be used for a Percutaneous Coronary Intervention (PCI); and (ii) in a case where the interventional device is a stent, perform one or more of: detecting stent expansion or underexpansion, detecting stent apposition or malapposition, performing co-registration, performing imaging, displaying a notification regarding the detected stent expansion or underexpansion, displaying a notification regarding the detected stent apposition or malapposition, and confirming stent placement.

In one or more embodiments, one or more processors may employ computational fluid dynamics (CFD). For example, one or more embodiments of the present disclosure may employ information on 2D or 3D results and/or structure(s) for the object in order to construct a CFD model for the object.

In one or more embodiments of the present disclosure, at least one method for calculating or deriving FFR measurements (and at least one storage medium having one or more programs stored therein that operate to cause a computer or processor to perform a method(s), where the method) may include: (i) fully and automatically calculating an FFR using the obtained images of at least one imaging modality (e.g., OCT, OCT only, IVUS, IVUS only, NIRF, NIRF only, NIRAF, NIRAF only, any other imaging modality discussed herein, etc.); (ii) automatically calculating one or more arterial branches; (iii) calculating (e.g., manually or automatically) an arterial pressure loss due to the arterial branches; and/or (iv) deriving one or more patient specific FFR measurements.

In one or more embodiments of the present disclosure, at least one method for calculating or deriving FFR measurements (and at least one storage medium having one or more programs stored therein that operate to cause a computer or processor to perform a method(s), where the method) may include: (i) obtaining intravascular image data (e.g., for a pullback); (ii) detecting lumen area(s) using a lumen detection method or technique; (iii) detecting a minimum lumen area (As) and define a stenotic area (L); (iv) constructing a carpet view (e.g., of the pullback) and automatically calculating the area(s) of any arterial branch(es); (v) in a case where an arterial branch is within (or has a portion that passes through or is within) the stenotic area, reducing a velocity of a fluid (e.g., blood) or object passing through the branch or lumen; (vi) calculating a diastolic and systolic (e.g., of a patient, of a specific patient, for one or more patients, for an object or sample, etc.) Stenotic Flow Reserve(s) (SFR) using the velocity, the stenotic area (L), and the minimum lumen area (As); and/or (vii) using the SFR, calculating the Fractional Flow Reserve (FFR).

The present disclosure describes a means to allow OCT users to focus on the area of interest in one or more imaging modalities, such as, but not limited to, the aforementioned imaging modalities, any other imaging modalities discussed herein, etc.

As described herein, one or more embodiments of the present disclosure may provide at least one imaging or optical apparatus/device, system, method, and storage medium that may use one or more imaging modalities and that may use one or more FFR calculation processes or techniques that operate to reduce both the cost and the interventional risk during PCI procedure(s).

When the user obtains an intravascular image at a location within the object, that specific portion of the object may be at a predetermined location based on prior angiographic images or other information.

While more than one angiography image or intravascular image may be used in one or more embodiments of the present disclosure, at least one intravascular image may be used in one or more embodiments.

Turning now to the details of the figures, imaging modalities may be displayed and/or FFR may be calculated in one or more ways as discussed herein. One or more displays discussed herein may allow a user of the one or more displays to use, control and/or emphasize multiple imaging techniques or modalities, such as, but not limited to, OCT, NIRAF, etc., may allow the user to use, control, and/or emphasize the multiple imaging techniques or modalities synchronously, and may allow the user to calculate or derive FFR more accurately (as compared to cases where the user would not be using the method(s) or technique(s) of the present disclosure).

As shown diagrammatically in FIG. 1A, one or more embodiments for calculating or determining/deriving FFR or FFR measurements of the present disclosure may be involved with one or more predetermined or desired procedures, such as, but not limited to, medical procedure planning and performance (e.g., PCI as aforementioned). For example, the system 2 may communicate with the image scanner 5 (e.g., a CT scanner, an X-ray machine, etc.) to request information for use in the medical procedure (e.g., PCI) planning and/or performance, such as, but not limited to, bed positions, and the image scanner 5 may send the requested information along with the images to the system 2 once a clinician uses the image scanner 5 to obtain the information via scans of the patient. In some embodiments, one or more angiograms 3 taken concurrently or from an earlier session are provided for further planning and visualization. The system 2 may further communicate with a workstation such as a Picture Archiving and Communication System (PACS) 4 to send and receive images of a patient to facilitate and aid in the medical procedure planning and/or performance. Once the plan is formed, a clinician may use the system 2 along with a medical procedure/imaging device 1 (e.g., an imaging device, an OCT device, an IVUS device, a PCI device, an ablation device, an FFR determination or calculation device, etc.) to consult a medical procedure chart or plan to understand the shape and/or size of the targeted biological object to undergo the imaging and/or medical procedure. Each of the medical procedure/imaging device 1, the system 2, the locator device 3, the PACS 4 and the scanning device 5 may communicate in any way known to those skilled in the art, including, but not limited to, directly (via a communication network) or indirectly (via one or more of the other devices such as 1 or 5, or additional flush and/or contrast delivery devices; via one or more of the PACS 4 and the system 2; via clinician interaction; etc.).

In medical procedures, improvement or optimization of physiological assessment is preferable to decide a course of treatment for a particular patient. By way of at least one example, physiological assessment is very useful for deciding treatment for cardiovascular disease patients. In a catheterization lab, for example, physiological assessment may be used as a decision-making tool—e.g., whether a patient should undergo a PCI procedure, whether a PCI procedure is successful, etc. While the concept of using physiological assessment is theoretically sound, physiological assessment still waits for more adaption and improvement for use in the clinical setting(s). This situation may be because physiological assessment may involve adding another device and medication to be prepared, and/or because a measurement result may vary between physicians due to technical difficulties. Such approaches add complexities and lack consistency. Therefore, one or more embodiments of the present disclosure may employ CFD-based physiological assessment that may be performed from imaging data to eliminate or minimize technical difficulties, complexities and inconsistencies during the measurement procedure (e.g., one or more methods of the present disclosure may use 2D or 3D results and/or 2D or 3D structure(s) and may calculate or derive the FFR; one or more methods of the present disclosure may calculate the FFR and provide information on treatment option(s) for the treatment of stenosis and/or another medical condition; one or more methods of the present disclosure may employ information on 2D or 3D results and/or structure(s) for the object in order to construct a CFD model for the object; one or more methods of the present disclosure may employ CFD to calculate one or more pressures and to have or obtain the FFR; one or more methods of the present disclosure may calculate FFR and may automatically decide or a user may decide to treat or not treat stenosis and/or other condition; one or more methods of the present disclosure may use FFR in real-time; one or more methods of the present disclosure may calculate pressure(s) and may include a lamp parameter/circuit analog model; one or more embodiments of the present disclosure may include an OCT FFR method that uses anatomic information (e.g., a volume of a vessel, any other anatomic information discussed in the present disclosure, arterial pressure(s), etc.); etc.). To obtain accurate physiological assessment, an accurate 2D or 3D structure of the vessel may be reconstructed from the imaging data, and/or an accurate FFR may be calculated or derived.

In at least one embodiment of the present disclosure, method(s) or technique(s) may be used to calculate or derive a more accurate FFR (compared to scenario(s) not using the method(s) or technique(s) of the present disclosure). In one or more embodiments, a combination of multiple imaging modalities may be used via adding another specific imaging condition for physiological assessment. In at least one further embodiment example, a method of FFR calculation without adding any imaging requirements or conditions may be employed. One or more methods of the present disclosure may use intravascular imaging, e.g., IVUS, OCT, etc., and one (1) view of angiography. One or more methods or embodiments of the present disclosure may use at least one intravascular image or view. In the description below, while intravascular imaging of the present disclosure is not limited to OCT, OCT is used as a representative of intravascular imaging for describing one or more features herein.

Referring now to FIG. 1B, shown is a schematic diagram of at least one embodiment of an imaging system 20 for generating an imaging catheter path (e.g., based on either a directly detected location of a radiopaque marker on the imaging catheter or a regression line representing the imaging catheter path by using an angiography image frame that may be simultaneously acquired during intravascular imaging pullback). The imaging system 20 may include an angiography system 30, an intravascular imaging system 40, an image processor 50, a display or monitor 1209, and an electrocardiogramaevice 60. The angiography system 30 may include an X-ray imaging device such as a C-arm 22 that is connected to an angiography system controller 24 and an angiography image processor 26 for acquiring angiography image frames of an object, sample, or patient 106.

The intravascular imaging system 40 of the imaging system 20 may include a console 32, a catheter 120 and a patient interface unit or PIU 110 that connects between the catheter 120 and the console 32 for acquiring intravascular image frames. The catheter 120 may be inserted into a blood vessel of the object, sample, or patient 106. The catheter 120 may function as a light irradiator and a data collection probe that is disposed in the lumen of a particular blood vessel, such as, for example, a coronary artery. The catheter 120 may include a probe tip, one or more radiopaque markers, an optical fiber, and a torque wire. The probe tip may include one or more data collection systems. The catheter 120 may be threaded in an artery of the object, sample, or patient 106 to obtain images of the coronary artery. The patient interface unit 110 may include a motor M inside to enable pullback of imaging optics during the acquisition of intravascular image frames. The imaging pullback procedure may obtain images of the blood vessel. The imaging pullback path may represent the co-registration path, which may be a region of interest or a targeted region of the vessel.

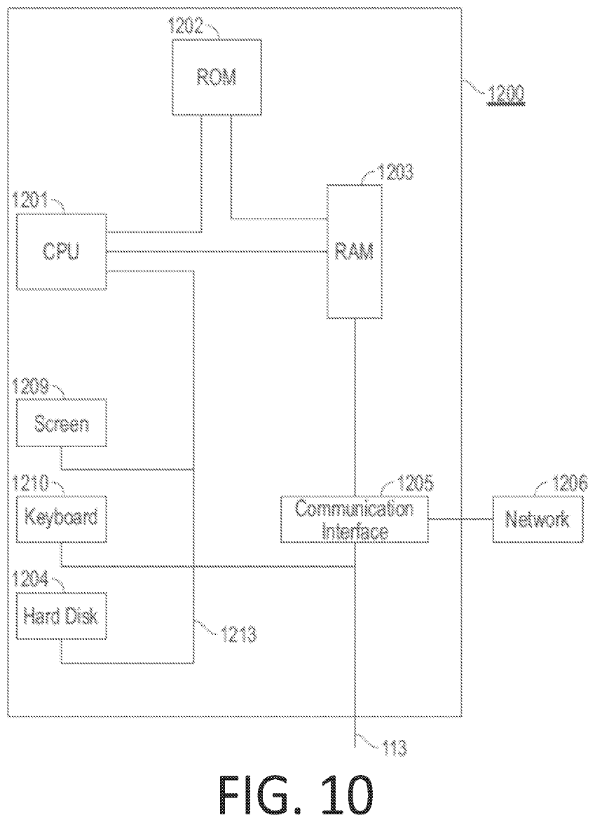

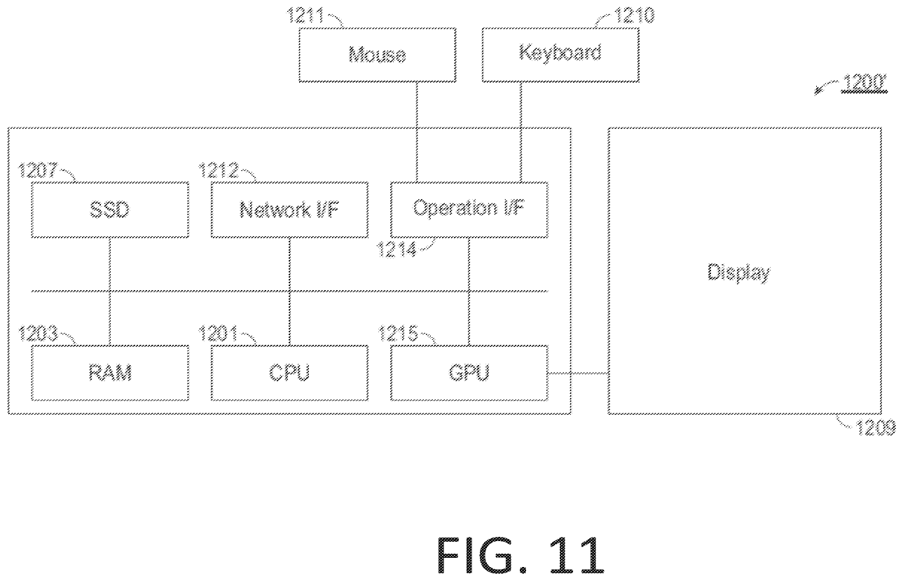

The console 32 may include a light source(s) 101 and a computer 1200. The computer 1200 may include features as discussed herein and below (see e.g., FIG. 10), or alternatively may be a computer 1200′ (see e.g., FIG. 11) or any other computer or processor discussed herein. In one or more embodiments, the computer 1200 may include an intravascular system controller 35 and an intravascular image processor 36. The intravascular system controller 35 and/or the intravascular image processor 36 may operate to control the motor M in the patient interface unit 110. The intravascular image processor 36 may also perform various steps for image processing and control the information to be displayed.

Various types of intravascular imaging systems may be used within the imaging system 20. The intravascular imaging system 40 is merely one example of an intravascular imaging system that may be used within the imaging system 20. Various types of intravascular imaging systems may be used, including, but not limited to, an OCT system, a multi-modality OCT system or an IVUS system, by way of example.

The imaging system 20 may also connect to an electrocardiogramaevice 60 for recording the electrical activity of the heart over a period of time using electrodes placed on the skin of the patient 106. The imaging system 20 may also include an image processor (e.g., the computer or processor 1200 discussed herein, the computer or processor 1200′ discussed herein, the computer 2 discussed herein, the image processor 50 shown in FIG. 1B, etc.) for receiving angiography data, intravascular imaging data, and data from the ECG device 60 to execute various image-processing steps to transmit to a display 1209 for displaying an angiography image frame with a co-registration path and/or for displaying an intravascular image. Although the image processor 50 associated with the imaging system 20 appears external to both the angiography system 20 and the intravascular imaging system 30 in FIG. 1B, the image processor 50 may be included within the angiography system 30, the intravascular imaging system 40, the display 1209, or a stand-alone device. Alternatively, the image processor 50 may not be required if the various image processing steps are executed using one or more of the angiography image processor 26, the intravascular image processor 36 of the imaging system 20, or any other processor discussed herein (e.g., computer 1200, computer 1200′, computer or processor 2, etc.).

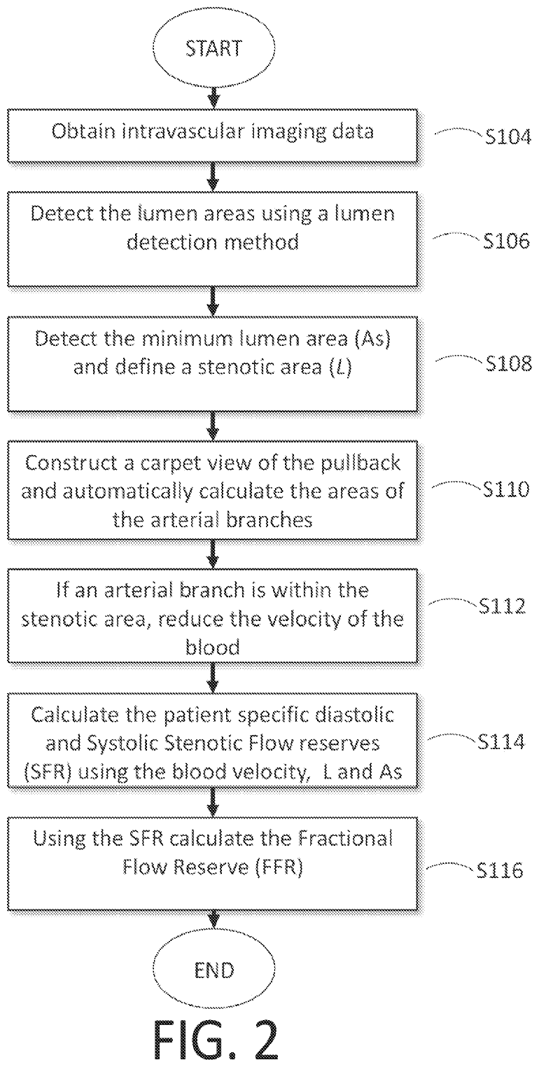

FIG. 2 shows at least one embodiment of workflow or overall workflow for one or more FFR calculation or derivation methods or techniques.

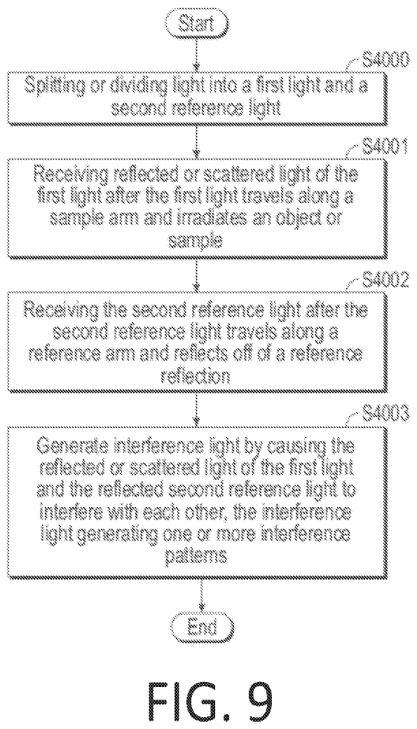

While not limited to the discussed combination or arrangement, one or more steps may be involved in the workflows or processes in one or more embodiments of the present disclosure, for example, as shown in FIG. 2 and/or FIG. 9 and as discussed below.

Returning to the details of FIG. 2, one or more methods or processes of the present disclosure may include one or more of the following steps: (i) obtaining intravascular image data (e.g., for a pullback) (see step S104 in FIG. 2); (ii) detecting lumen area(s) using a lumen detection method or technique (see step S106 in FIG. 2); (iii) detecting a minimum lumen area (As) and define a stenotic area (L) (see step S108 in FIG. 2); (iv) constructing a carpet view (e.g., of the pullback) and automatically calculating the area(s) of any arterial branch(es) (see step S110 in FIG. 2); (v) in a case where an arterial branch is within (or has a portion that passes through or is within) the stenotic area, reducing a velocity of a fluid (e.g., blood) or object passing through the branch or lumen (see step S112 in FIG. 2); (vi) calculating a diastolic and systolic (e.g., of a patient, of a specific patient, for one or more patients, for an object or sample, etc.) Stenotic Flow Reserve(s) (SFR) using the velocity, the stenotic area (L), and the minimum lumen area (As) (see step S114 in FIG. 2); and/or (vii) using the SFR, calculating the Fractional Flow Reserve (FFR) (see step S116 in FIG. 2).

Intravascular image data (see e.g., step S104 for FIG. 2) may be obtained using one or more probes, including the probes or catheters (see e.g., the catheter 120) discussed herein.

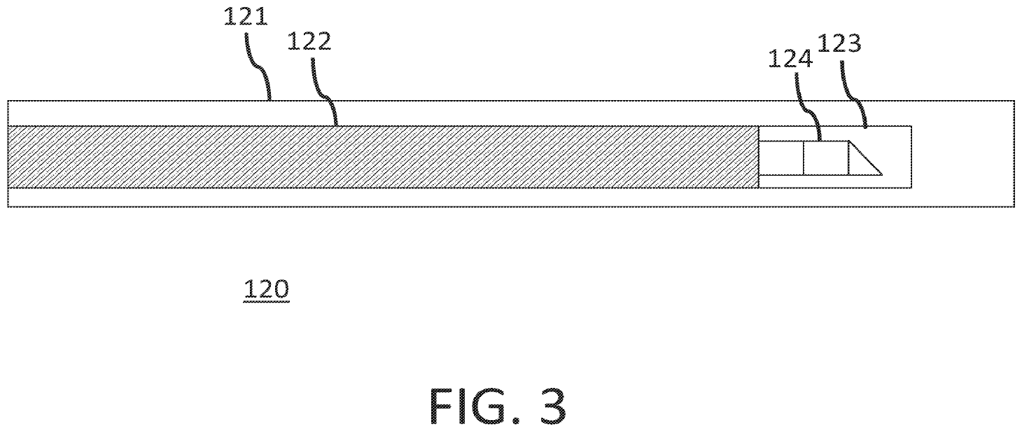

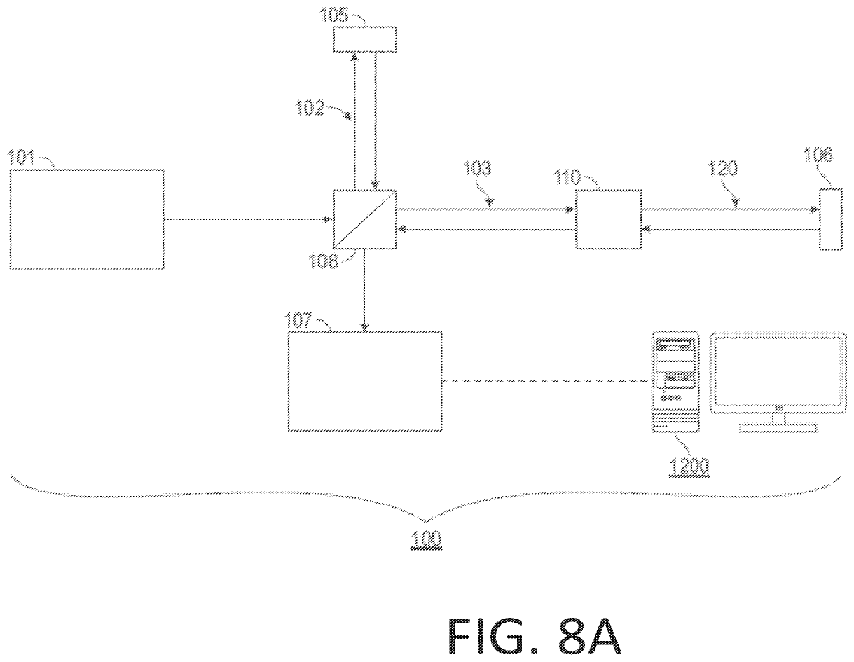

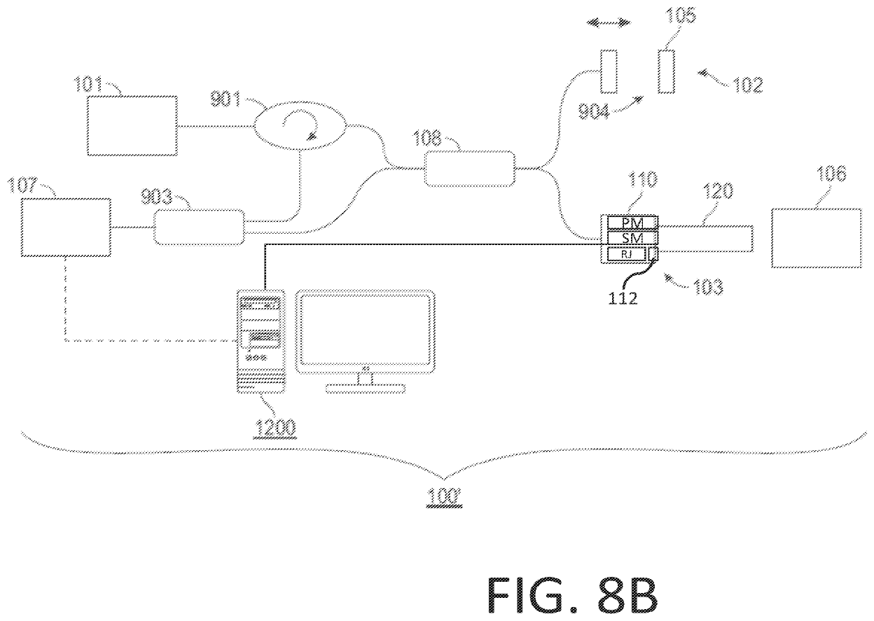

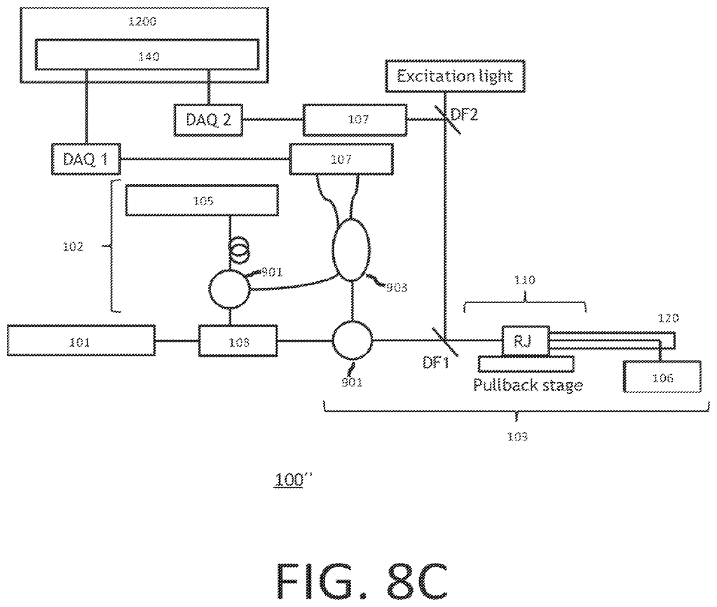

FIG. 3 shows at least one embodiment of a catheter 120 that may be used in one or more embodiments of the present disclosure to obtain images or imaging data, to construct or reconstruct 2D or 3D structure(s), and/or to operate for obtaining or calculating FFR or FFR measurements. FIG. 3 shows an embodiment of the catheter 120 including a sheath 121, a coil 122, a protector 123 and an optical probe 124. As shown schematically in FIGS. 8A-8C (discussed further below), the catheter 120 may be connected to a patient interface unit (PIU) 110 to spin the coil 122 with pullback (e.g., at least one embodiment of the PIU 110 operates to spin the coil 122 with pullback). The coil 122 delivers torque from a proximal end to a distal end thereof (e.g., via or by a rotational motor in the PIU 110). In one or more embodiments, the coil 122 is fixed with/to the optical probe 124 so that a distal tip of the optical probe 124 also spins to see an omnidirectional view of the object (e.g., a biological organ, sample or material being evaluated, such as, but not limited to, hollow organs such as vessels, a heart, a coronary artery, etc.) or the patient. For example, fiber optic catheters and endoscopes may reside in the sample arm (such as the sample arm 103 as shown in one or more of FIGS. 8A-8C discussed below) of an OCT interferometer in order to provide access to internal organs, such as intravascular images, gastro-intestinal tract or any other narrow area, that are difficult to access. As the beam of light through the optical probe 124 inside of the catheter 120 or endoscope is rotated across the surface of interest, cross-sectional images of one or more objects are obtained. In order to acquire three-dimensional data, the optical probe 124 is simultaneously translated longitudinally during the rotational spin resulting in a helical scanning pattern. This translation is most commonly performed by pulling the tip of the probe 124 back towards the proximal end and therefore referred to as a pullback.

The catheter 120, which, in one or more embodiments, comprises the sheath 121, the coil 122, the protector 123 and the optical probe 124 as aforementioned (and as shown in FIG. 3), may be connected to the PIU 110. In one or more embodiments, the optical probe 124 may comprise an optical fiber connector, an optical fiber and a distal lens. The optical fiber connector may be used to engage with the PIU 110. The optical fiber may operate to deliver light to the distal lens. The distal lens may operate to shape the optical beam and to illuminate light to the object (e.g., the object 106 (e.g., a vessel) discussed herein), and to collect light from the sample (e.g., the object 106 (e.g., a vessel) discussed herein) efficiently.

As aforementioned, in one or more embodiments, the coil 122 delivers torque from a proximal end to a distal end thereof (e.g., via or by a rotational motor in the PIU 110). There may be a mirror at the distal end so that the light beam is deflected outward. In one or more embodiments, the coil 122 is fixed with/to the optical probe 124 so that a distal tip of the optical probe 124 also spins to see an omnidirectional view of an object (e.g., a biological organ, sample or material being evaluated, such as, but not limited to, hollow organs such as vessels, a heart, a coronary artery, etc.). In one or more embodiments, the optical probe 124 may include a fiber connector at a proximal end, a double clad fiber and a lens at distal end. The fiber connector operates to be connected with the PIU 110. The double clad fiber may operate to transmit & collect OCT light through the core and, in one or more embodiments, to collect Raman and/or fluorescence from an object (e.g., the object 106 (e.g., a vessel) discussed herein, an object and/or a patient (e.g., a vessel in the patient), etc.) through the clad. The lens may be used for focusing and collecting light to and/or from the object (e.g., the object 106 (e.g., a vessel) discussed herein). In one or more embodiments, the scattered light through the clad is relatively higher than that through the core because the size of the core is much smaller than the size of the clad.

In one or more embodiments, one or more lumen detection method(s) may be used, such as, but not limited to, the lumen detection method(s) as discussed in U.S. Pat. Pub. No. 2021/0407098 A1, published Dec. 30, 2021, the entirety of which is incorporated by reference. Additionally or alternatively, in one or more embodiments, a lumen or lumens may be detected manually (in addition to or alternatively to an automatic method).

In one or more embodiments, a minimum lumen may be determined or calculated automatically, and a normal area may be extracted.

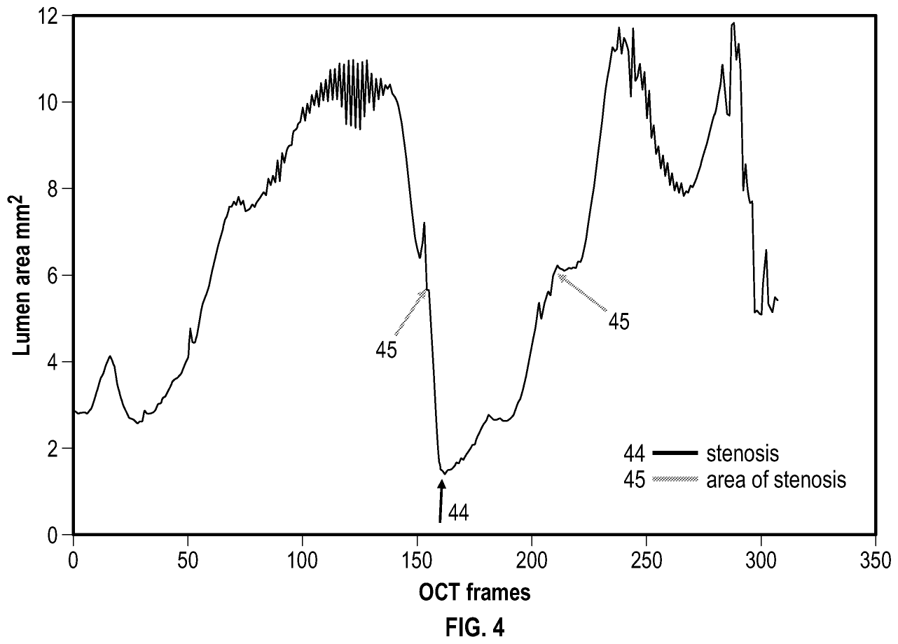

In one or more embodiments of at least one method or technique, or an apparatus or storage medium for use with same, a stenotic area of a pullback (e.g., an OCT pullback, a pullback of another imaging modality, etc.) may be found (see e.g., step S108 of FIG. 2) (see element 44 in FIG. 4). For example, in one or more embodiments a minimum lumen area (“As”) (e.g., a minimum OCT lumen area, a minimum lumen area of another imaging modality, etc.) may be detected. In one or more embodiments, the highest peak (e.g., that drops for the next five (5) frames in one or more embodiments, a peak surrounded or bookended by drops on both sides, etc.) after the stenosis may be marked as a distal part or peak of the stenosis. Then, a first part or peak may be determined before the stenosis where the first part or peak has the same or similar value to the distal part or peak of the stenosis. The first part or peak is the proximal part or peak of the stenosis and, together with the distal part or peak, the subject peaks define the stenotic area which as a length “L”. The first and second parts or peaks of the stenosis (see elements 45 in FIG. 4). “An” is defined as an area of a lowest stenotic peak.

In one or more embodiments, a calculation of an imaging modality-derived (e.g., OCT-derived, IVUS-derived, etc.) pressure loss may be calculated. For example, Gould et al. (K. L. Gould, K. O. Kelley, and E. L. Bolson, “Experimental validation of quantitative coronary arteriography for determining pressure-flow characteristics of coronary stenosis,” Circulation, vol. 66, no. 5 I, pp. 930-937, 1982, doi: 10.1161/01.CIR.66.5.930, which is incorporated by reference herein in its entirety; hereinafter referred to as the “first Gould et al. reference”) showed that a calculated pressure drop (AP) across a stenosis may be described, in one or more embodiments, by the following simplified equation:

$$Δ P = FV + {SV}^{2} , F = \frac{8 πμ L}{As} \frac{An}{As} \& S = \frac{ρ}{2} {( \frac{An}{As} - 1 )}^{2} , \qquad ( 1 )$$

where μ=blood viscosity, L=stenosis length, An=cross-sectional area of the normal artery or the area with the lowest stenotic peak (or an average of the lowest points 45 in FIG. 4 in one or more embodiments), As=cross-sectional area of the stenosis segment (or the reference lumen area), V=flow velocity, ρ=blood density, F and S=the coefficients of pressure drop due to viscous friction and exit separation, respectively. Resistance may be calculated from IVI geometry for both Poiseuille resistance due to viscous friction (F), assuming laminar flow in the converging portion of the stenosis, and for resistance due to exit separation(S) due to vortex formation in the diverging portion of the stenosis.

In one or more embodiments, the coefficients may be described as μ=4.0×10−3 Pa·s, and ρ=1050 kg/m3 (see e.g., the first Gould et al. reference). The sum of resistance of all frames in the lesion may be equivalent to the Poiseuille resistance of the entire lesion in one or more embodiments. The coefficient of S may be calculated using the reference lumen area and minimum lumen area (MLA). In one or more embodiments, an MLA may be defined by the stenosis (see element 44 in FIG. 4). In one or more embodiments, a reference lumen area may be defined by elements 45 (e.g., may be a mean or average of the values at points 45 in FIG. 4) for the “area of stenosis” as shown in FIG. 4. In one or more embodiments, the cross-sectional area of the normal artery may be the same as the reference lumen area.

$$F = \frac{8 πμ {L}_{s}}{As} \frac{An}{As} , \qquad ( 2 )$$

where As is a stenotic area (e.g., area between both elements 45 in FIG. 4 in at least one embodiment), Ls is stenotic length, As′ may be located in any area and not necessarily in the stenotic area and As′ may be a lowest area of a stenotic area or areas. In one or more embodiments, the variables or terms of equation (2) may be defined as used in the Gould reference.

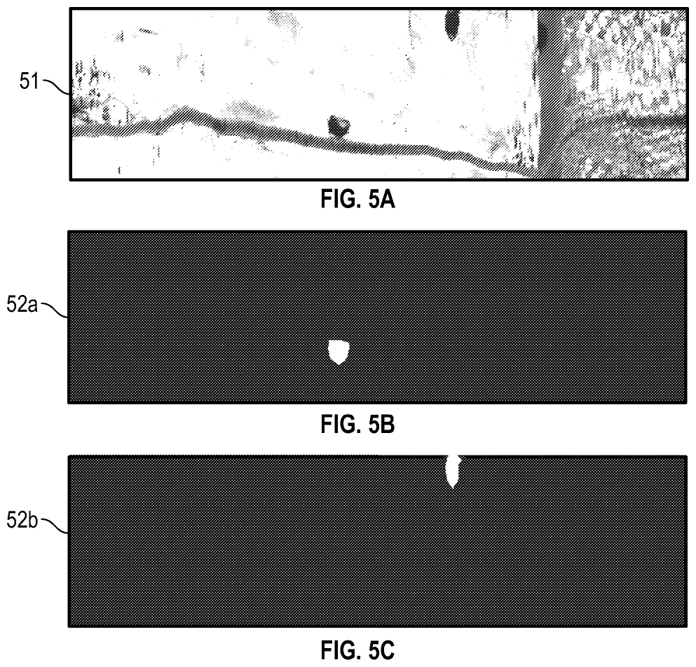

In one or more embodiments, an arterial branch FFR adjustment may be performed. In one or more embodiments, a carpet view of the pullback may be constructed (see e.g., step S110 in FIG. 2), and the areas of the arterial branches may be calculated. To automatically adjust the FFR according to an arterial branch pressure loss or losses, the area of each branch may be calculated. Manual branch detection may be performed in one or more embodiments. However, since manual branch detection may be time consuming and not accurate enough in one or more situations, an automated method for branch detection may be performed as discussed herein, and the method may create the pullback carpet view (see carpet view 51 in FIG. 5A), detect the branches (see views 52a and 52b in FIGS. 5B and 5C, respectively, and calculate the area of the branches as schematically shown in FIGS. 5A-5C.

In one or more embodiments of step S112 of FIG. 2, it may be determined whether an arterial branch or branches (or a portion or portions of the branch or branches) is/are within the stenotic area. In a case where a detected arterial branch is within the stenotic area L, the step S112 may calculate a percentage Per of the branch area when/as being compared to the normal area An, and then may recalculate, the flow V as being equal to V×Per such that V=V*Per.

In one or more embodiments of step S114 of FIG. 2, a calculation of a Stenotic Flow Reserve or Reserves may be performed. In one or more embodiments, a modified Stenotic Flow Reserve (SFR) may be calculated using a mathematical circulation model of a microvascular pressure loss, which subtracts a pressure drop of the epicardial artery from the aortic pressure. One or more embodiments may calculate the modified SFR in this way as discussed, for example, in at least B. De Bruyne et al., “Fractional Flow Reserve—Guided PCI versus Medical Therapy in Stable Coronary Disease,” N. Engl. J. Med., vol. 367, no. 11, pp. 991-11 Sep. 2012, doi: 10.1056/NEJMoa1205361, which is incorporated by reference herein in its entirety, and/or as discussed in at least B. De Bruyne et al., “Fractional Flow Reserve-Guided PCI for Stable Coronary Artery Disease,” N. Engl. J. Med., vol. 371, no. 13, pp. 1208-1217 September 2014, doi: 10.1056/NEJMOA1408758/SUPPL_FILE/NEJMOA1408758_DISCLOSURES.PDF, which is incorporated by reference herein in its entirety. In other words, in one or more embodiments, SFR may be the coronary flow reserve calculated based on the coronary anatomical measurements. One or more ways to calculate SFR are further discussed in R. L. Kirkeeide, K. L. Gould, and L. Parsel, “Assessment of coronary stenoses by myocardial perfusion imaging during pharmacologic coronary vasodilation. VII. Validation of coronary flow reserve as a single integrated functional measure of stenosis severity reflecting all its geometric dimensions,” J. Am. Coll. Cardiol., vol. 7, no. 1, pp. 103-113, January 1986, doi: 10.1016/S0735-1097(86)80266-2 (hereinafter referred to as “the second Gould et al. reference”), which is incorporated by reference herein in its entirety, and in the first Gould et al. reference, which is incorporated by reference herein in its entirety.

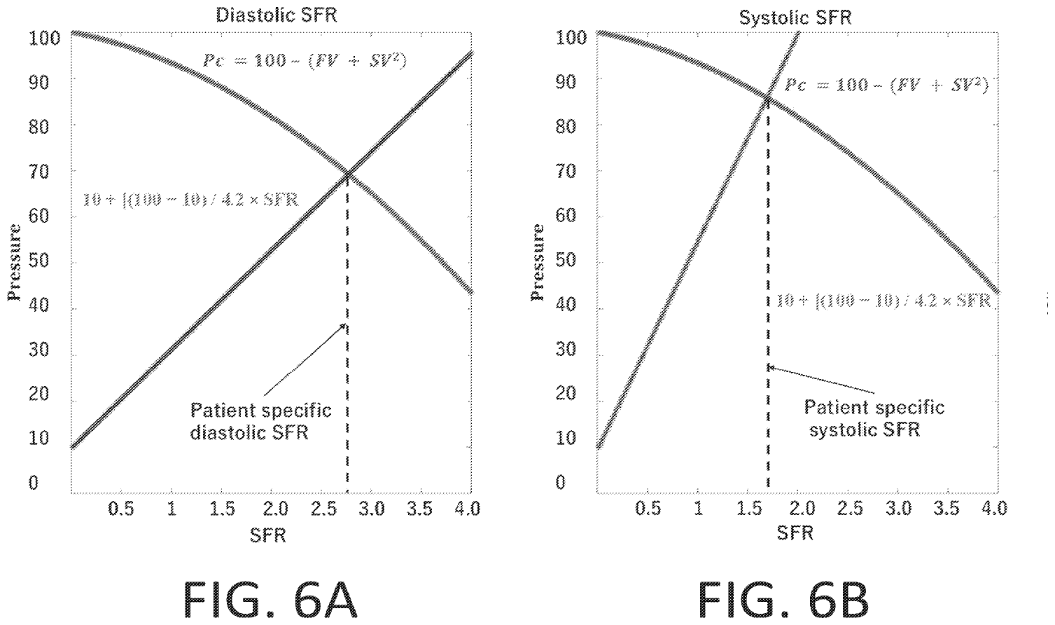

In one or more embodiments, it may be assumed that an object, sample, or patient has a mean arterial pressure of 100 mm Hg and a coronary flow that may increase to 4.2 times its value at rest without stenosis. Under such conditions, one or more embodiments may then graphically determine the SFR by plotting coronary pressure against relative coronary flow. One or more ways of determining SFR are further discussed in the first and second Gould et al. references. A maximum SFR was determined as of, about, or as being 4.2 (see e.g., discussions in A. Jeremias et al., “Effects of Intravenous and Intracoronary Adenosine 5′-Triphosphate as Compared With Adenosine on Coronary Flow and Pressure Dynamics,” Circulation, vol. 101, no. 3, pp. 318-323, January 2000, doi: 10.1161/01.CIR.101.3.318, which is incorporated by reference herein in its entirety, and in J. A. Leppo, “Comparison of pharmacologic stress agents,” J Nucl Cardiol., November-December 1996, 3(6 Pt 2):S22-6, doi: 10.1016/s1071-3581(96)90204-4, which is incorporated by reference herein in its entirety. As shown in the embodiment example of FIGS. 6A-6B, the intersection of the curve (100-ΔP) with the line representing coronary perfusion pressure under hyperemia is the SFR of that region for the flow in the distal coronary vascular bed under conditions of hyperemia. Coronary flow concepts are further discussed in H. Wieneke et al., “Corrected coronary flow velocity reserve: a new concept for assessing coronary perfusion,” J. Am. Coll. Cardiol., vol. 35, no. 7, pp. 1713-1720 June 2000, doi: 10.1016/S0735-1097(00)00639-2, which is incorporated by reference herein in its entirety. The SFR value was calculated using the following formulas:

$$Pc = 100 - Δ P = 100 - ( FV + SV 2 ) , \qquad ( 3 )$$

where Pc (coronary pressure distal to the stenosis) is defined as the translesional pressure. As shown in FIGS. 6A-6B, Pc is plotted on the vertical axis, and coronary artery velocity (V) is plotted on the horizontal axis as a ratio of velocity at hyperemia to velocity at normal flow at rest (Vhyperemia/Vrest=SFR). In the SFR calculation, a coronary flow velocity of 0.2 m/s results in an SFR value of 1, and a coronary flow velocity value of 0.4 m/s produces an SFR value of 2. The positively sloped line in FIGS. 6A and 6B plots the relationship between coronary perfusion pressure and coronary flow under conditions of maximum coronary vasodilation in the presence of a stenosis according to the equation: 10+[(100−10)/4.2×SFR (see FIGS. 6A-6B). The negatively sloped curve is a plot of the relation between Pc and flow in the presence of a stenosis. This solid curve is the graphic plot of the equation at the bottom of the figure derived from the equation: 100−ΔP=100−(FV+SV²). The intersection of the curve with the line representing coronary perfusion pressure under hyperemia is the lesion-specific SFR (see FIGS. 6A-6B).

In one or more embodiments, basal (diastolic/systolic) LAD flow may be determined as being 20/10 cm/sec, and basal (diastolic/systolic) RCA flow may be determined as being 15/10 cm/sec in one or more embodiments of the method(s) or algorithm(s) of the present disclosure. Corrected coronary flow velocity is further discussed in H. Wieneke et al., “Corrected coronary flow velocity reserve: a new concept for assessing coronary perfusion,” J. Am. Coll. Cardiol., vol. 35, no. 7, pp. 1713-1720 June 2000, doi: 10.1016/S0735-1097(00)00639-2, which is incorporated by reference herein in its entirety. Maximum flow velocity (V) may be calculated as “basal coronary flow velocity×SFR.” The flow velocity V may be calculated for the systole/diastole as:

$$V = SFR * Vds , \qquad ( 4 )$$

where Vds is a fixed value for the diastole and systole: 20/15 (Left/right coronary) cm/sec and 10 (Left/right coronary) cm/sec, respectively. After the Vs (for the systole) flow velocity value and the Vd for the diastole flow velocity value are calculated, then the ΔPₛ (pressure drop or change for the systole velocity value) and ΔP_{d} (pressure drop or change for the diastole velocity value) using Eq. 1 above may be calculated.

In one or more embodiments, a systolic/diastolic (mean) blood pressure may be or have a value of 120/60 (systolic/diastolic) (80) mm Hg. A discussion of mean blood pressure may be found in M. Miyagawa et al., “Thallium-201 myocardial tomography with intravenous infusion of adenosine triphosphate in diagnosis of coronary artery disease,” J. Am. Coll. Cardiol., vol. 26, no. 5, pp. 1196-121 November 1995, doi: 10.1016/0735-1097(95)00304-5, which is incorporated by reference herein in its entirety. The proportion of diastolic time may be determined as ⅔ of the whole cardiac cycle in one or more embodiments. The LCX pressure loss value during diastole/systole is defined, for one or more embodiments, as the mean of each diastole/systole of RCA and LAD. Then, the FFR value may be calculated as:

$$FFR = \frac{( 60 - Δ {P}_{d} ) * \frac{2}{3} + ( 120 - Δ {P}_{s} ) * \frac{1}{3}}{80} . \qquad ( 5 )$$

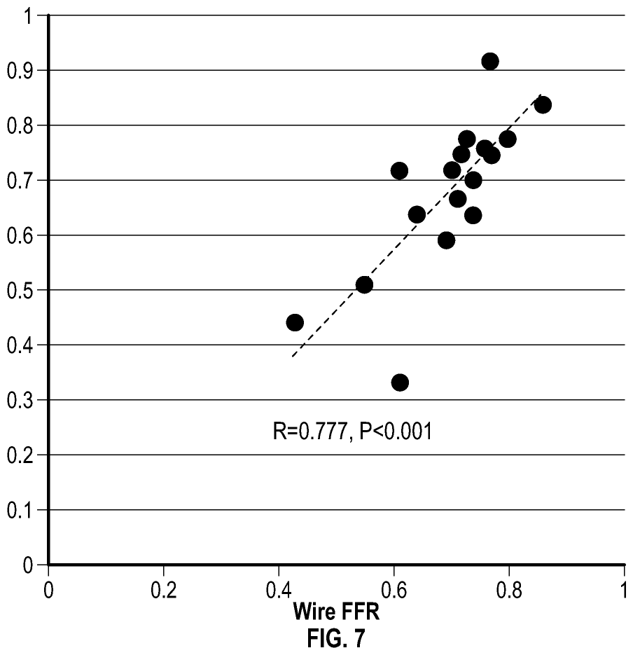

Seventeen (17) FFR wire based measurements were used to test the validity and accuracy of the FFR calculation measurements, techniques, algorithms, and methods of the present disclosure. The FFR calculation measurements, techniques, algorithms, and methods of the present disclosure have a good/moderate (R=0.777) agreement with the wire based measurements, and the results of the test comparison are presented in FIG. 7. The FFR numbers may be reproduced using the one or more methods of the present disclosure. As such, the one or more FFR calculation methods of the present disclosure provide a useful alternative to other wire based measurements, and also the methods of the present disclosure provide the numerous other advantages and features discussed herein.

In one or more embodiments using intravascular and angiography images, such intravascular and angiography images may be obtained simultaneously or independently at different times. It is understood that any simultaneous acquisition of, for example, an angiographic image and an OCT intravascular image may be performed at a different amount of time (e.g., milliseconds compared to several seconds). Thus, the term ‘simultaneous’ includes an angiographic image (or multiple angiographic images) taken at any time during an OCT pullback. While one or more steps may be performed to simultaneously acquire an intravascular image and an angiography image in one or more embodiments, such image acquisition may be performed at different times (or not being simultaneously acquired) in one or more other embodiments, such as, but not limited to, embodiment(s) as discussed in U.S. Pat. App. No. 62/798,885, filed on Jan. 30, 2019, the application of which is incorporated by reference herein in its entirety. Indeed, co-registration may be performed under either scenario. In one or more embodiments where an angiography image is acquired simultaneously with an intravascular image, the one or more such embodiments may increase the accuracy of the co-registration because a radiopaque marker location, which is the acquisition location of an intravascular (e.g., OCT) image, may be detected. In one or more embodiments, OCT/IVUS and angiography modalities are available when using images that are acquired during a procedure (e.g., a PCI procedure). In one or more embodiments, where a CT image is acquired prior to the PCI procedure, co-registration between CT and angiography, and/or between CT and OCT/IVUS, may be performed. Using CT and OCT/IVUS is further discussed in U.S. Pat. Pub. No. 2018/0271614, which publication is incorporated by reference herein in its entirety. While one or more PCI procedures discussed herein discuss stent implantation, balloon angioplasty or other procedures in coronary arteries and other arteries (e.g., arteries located in one or more legs or other body parts), PCT procedures are not limited thereto. For example, in addition to uses for coronary procedures, OCT/IVUS may be used in other region(s) of vasculature. In one or more embodiments, a first set of angiography image(s) may be used for an initial analysis of an object, sample, or patient or the case, and a second set of angiography image(s) may be used for co-registration. The second angiography image(s) obtained may be obtained during OCT pullback to achieve more accurate co-registration.

Visualization, PCI procedure planning, and physiological assessment may be combined to perform complete PCI planning beforehand, and to perform complete assessment after the procedure. Once a 2D or 3D structure is constructed or reconstructed and a user specifies an interventional device, e.g., a stent, that is planned to be used, virtual PCI may be performed in a computer simulation (e.g., by one or more of the computers discussed herein, such as, but not limited to, the computer 2, the processor computer 1200, the processor or computer 1200′, any other processor discussed herein, etc.). Then, another physiological assessment may be performed based on the result of the virtual PCI. This approach allows a user to find the best device (e.g., interventional device, implant, stent, etc.) for each patient before or during the procedure.

In one or more additional or alternative embodiments, one or more other imaging modalities may be used, such as CT and/or magnetic resonance imaging (MRI), to define a curvature of an object (e.g., a vessel) instead of using an angiography image and/or instead of using another type of intravascular image. Since multiple slices may be captured with CT or MRI, a 3D structure of the object (e.g., a vessel) may be reconstructed from CT.

Graphical User Interface (GUI) features, imaging modality features, or other imaging features, may be used in one or more embodiments of the present disclosure, such as the GUI feature(s), imaging feature(s), and/or imaging modality feature(s) disclosed in U.S. patent Ser. No. 16/401,390, filed May 2, 2019, which was published as U.S. Pat. Pub. No. 2019/0339850 on Nov. 7, 2019, and disclosed in U.S. Pat. Pub. No. 2019/0029624 and WO 2019/023375, which application(s) and publication(s) are incorporated by reference herein in their entireties.

One or more other methods or algorithms may be used to confirm stent placement. For example, one or more methods or algorithms for calculating stent expansion/underexpansion or apposition/malapposition may be used in one or more embodiments of the present disclosure, including, but not limited to, the expansion/underexpansion and apposition/malapposition methods or algorithms discussed in U.S. Pat. Pub. Nos. 2019/0102906 and 2019/0099080, which publications are incorporated by reference herein in their entireties.

One or more methods or algorithms for calculating or evaluating cardiac motion using an angiography image and/or for displaying anatomical imaging may be used in one or more embodiments of the present disclosure, including, but not limited to, the methods or algorithms discussed in U.S. Pat. Pub. No. 2019/0029623 and U.S. Pat. Pub. No. 2018/0271614 and WO 2019/023382, which publications are incorporated by reference herein in their entireties.

One or more methods or algorithms for performing co-registration and/or imaging may be used in one or more embodiments of the present disclosure, including, but not limited to, the methods or algorithms discussed in U.S. Pat. App. No. 62/798,885, filed on Jan. 30, 2019 and published as WO 2020/159984, and discussed in U.S. Pat. Pub. No. 2019/0029624, which application(s) and publication(s) are incorporated by reference herein in their entireties.

For example, other options may be included in the GUI, such as, but not limited to, a Mark Slice feature, a Snapshot feature, an Annotation feature, etc. The Snapshot feature operates to take a snapshot or image of the current view of the GUI. The Annotation feature operates to allow a user of the GUI to include a comment(s) or note(s) for the viewed image or images. The Mark Slice feature allows the user to set points in a pullback feed of slices that are of interest (i.e., to mark a desired slice or slices).

Another option, in one or more embodiments, is a setting or feature icon or drop down menu that allows a user of the GUI to calculate one or more details of the image(s), such as, but not limited to, expansion/underexpansion (e.g., related to a reference area, of a stent, etc.), malapposition (e.g., of a stent, of a medical implant, etc.), etc. Information may be displayed to the right of the menu, such as, but not limited to, a percentage value of the reference area (e.g., “0-80% reference area” which indicates underexpansion exists in one or more embodiments and ma may be associated with a red box (or a box of a predetermined color) near or to the left of that information; “80-90% reference area” which may indicate that an issue may or may not exist (e.g., the underexpansion may fall within an acceptable range) related to underexpansion and may be associated with a yellow box (or a box of a predetermined color) near or to the left of that information, “90-100% reference area” which may indicate that an issue may not exist related to underexpansion and may be associated with a green box (or a box of a predetermined color) near or to the left of that information; etc.). Any colored box may be set at a predetermined location as desired in one or more embodiments. Such information and indicators may be used for apposition/malapposition in one or more embodiments. Additionally or alternatively, apposition/malapposition may be indicated with different predetermined ranges, such as, but not limited to, for example, greater than 300 microns (in other words, 300 microns or greater) may be used as the range for the red region or a region that needs or may need correction or action (e.g., a high risk region); between 200-300 microns may be used for the yellow region or a region that may need correction or action or to be watched closely or a region that is in an acceptable range to take no action or make no correction (e.g., a region between high and low risk, an acceptable region, etc.); less than 200 microns may be used for the green region or a region that has no issue detected and/or may require no action (e.g., a low risk region); etc. In one or more embodiments, different values or ranges may be assigned to the limits or ranges for the red or high risk region, the yellow or middle region and/or the green or acceptable region, for instance. The subject ranges may be decided by the apparatus, GUI, system, method, or storage medium automatically or may be selected by a user (e.g., a physician) manually. Depending on the application and use of the one or more embodiments of the present disclosure, such values may change accordingly. Other ranges may be designated for the high/low risk and/or acceptable or attention needed regions depending on the needs of a user and the medical procedure to be performed. Based on the data and associated warning or information displayed related to expansion/underexpansion and/or the apposition/malapposition, the GUI operates to indicate to a user of the GUI how to respond to that information (e.g., expansion/underexpansion and/or apposition/malapposition falls within an acceptable range such that no action may be needed; expansion/underexpansion and/or apposition/malapposition falls outside of an acceptable range such that action may be needed; expansion/underexpansion and/or apposition/malapposition falls in a range that requires correction or correction may be suggested; etc.). Any of the subject ranges (or any other range or ranges discussed in the present disclosure) may be selected manually or automatically as aforementioned. Such examples allow a user of the GUI to identify potential issues identified by the data in the one or more images, and may make appropriate decisions and create a plan accordingly.

Such information and other features discussed herein may be applied to other applications, such as, but not limited to, co-registration, FFR calculation, other modalities, etc. Indeed, the useful applications of the features of the present disclosure and of the aforementioned applications and patent publications are not limited to the discussed modalities, images, or medical procedures. Additionally, depending on the involved modalities, images, or medical procedures, one or more control bars may be contoured, curved, or have any other configuration desired or set by a user. For example, in an embodiment using a touch screen as discussed herein, a user may define or create the size and shape of a control bar based on a user moving a pointer, a finger, a stylus, another tool, etc. on the touch screen (or alternatively by moving a mouse or other input tool or device regardless of whether a touch screen is used or not). In one or more embodiments, such methods or techniques may be used to allow a user to interact with the carpet views or vessel or branch views (see e.g., FIGS. 5A-5C).

As aforementioned, one or more methods or algorithms for calculating expansion/underexpansion or apposition/malapposition may be used in one or more embodiments of the instant application, including, but not limited to, the expansion/underexpansion and apposition/malapposition methods or algorithms discussed in U.S. Pat. Pub. Nos. 2019/0102906 and 2019/0099080, which publications are incorporated by reference herein in their entireties. For example, in one or more embodiments for evaluating expansion/underexpansion, a method may be performed to remove inappropriate OCT image frames from the OCT image from further image processing. The result of lumen detection may be checked for each OCT image frame. If the lumen is not detected or if the detected lumen is affected by any artifact, the OCT image frame may be removed. A first OCT image frame is selected from the OCT image in a first step. After selecting the first OCT image frame, it may be determined whether a lumen is detected in the selected OCT image frame. If it is determined that no lumen has been detected in the OCT image frame, then the OCT image frame may be removed from further image processing and the process continues. Alternatively, if the lumen is detected in the frame, then a further determination of whether the detected lumen is affected by any artifact may be performed. If the detected lumen is affected by an artifact, then the OCT image frame may be removed from further processing and the process proceeds. If the detected lumen is not affected by any artifact, then it may be determined if the selected OCT image frame is the last OCT image frame from the OCT image. If the selected frame is not the last frame in the OCT image, then the next OCT image frame from the OCT image may be selected and the process returns to the lumen detection on the frame step. If the selected OCT image frame is the last OCT image frame, then the process proceeds. After removing the inappropriate OCT image frames, all the OCT image frames in which stent-struts are detected may be selected (Group Gₛ′). It may that the entire range of the stent region in the OCT image is going to be evaluated for stent expansion in one or more embodiments, but in another embodiment in this step a user may select one or more (first) ranges for evaluating stent expansion, from the stent region where the stent is implanted and the stent-struts are detected. Whether the user selects the first range as the entire range of the stent region or as a partial range of the entire stent region may depend upon system requirements or user needs. In one embodiment, the user may use a mouse device or touch screen device to designate one or more (first) ranges in the stent region, and a processor or CPU (e.g., the computer or processor 1200, 1200′, 2, etc. and/or any other processor discussed herein) may determine the first range for the stent expansion evaluation. This allows for designation of one or more positions. Subsequently, a reference OCT image frame based on the confirmed stented region may be selected. If the calculated stent length is equal to or within a predetermined threshold to the actual stent length, the OCT image frame at a position representing the distal end and the OCT image frame at a position representing the proximal end of the stented segment may be selected as reference frames. If the calculated stent length is not equal to the actual stent length and not within a predetermined threshold, the reference frames may be selected based on either the calculated stent length or the actual stent length. When the calculated stent length is selected for reference frame selection, the OCT image frame at a position representing the distal end and the OCT image frame at a position representing the proximal end of the stented segment may be selected as reference frames. Then, a reference OCT image frame may be selected based on the confirmed stented region. The reference area in the selected reference frame may be evaluated. Then, the first OCT image frame from the OCT image frames in which stent-struts are detected may be selected. Then the stent area is measured for the first OCT image frame. After measuring the stent area of the first OCT image frame, stent expansion may be evaluated by comparing the measured stent area and the reference area. The stent expansion value and an indicator for the corresponding stent expansion level may be saved with the first OCT image frame. After the stent expansion value is saved, it is determined whether the selected OCT image frame is the last frame. If the selected OCT image frame is not the last frame, then the next OCT image frame is selected and the process returns to the aforementioned measuring stent area step. In this example, because the selected OCT image frame is the first OCT image frame, the next frame would be the second OCT image frame from the group of all the OCT image frames in which stent-struts were detected. After selecting the next OCT image frame the process returns to the measure stent area step to measure the stent area for the next OCT image frame. Alternatively, if it is determined that the selected OCT image frame is the last frame, then the process for evaluating stent expansion is completed for the acquired OCT image. According to this workflow, every OCT image frame in which stent-struts are detected and not affected by artifact may be processed to obtain a stent expansion value based on the stent area associated with a selected OCT image frame and a reference area. In one or more embodiments, the reference area remains the same for each OCT image frame from the OCT image frames in which stent-struts are detected and not affected by artifact. By way of another example, in one or more embodiments for evaluating apposition/malapposition, a method may be performed to remove inappropriate OCT images as aforementioned. The result of lumen detection may be checked for each OCT image frame. If the lumen is not detected or if the detected lumen is affected by any artifact, the OCT image frame may be removed. A first OCT image frame is selected from the OCT image in a first step. After selecting the first OCT image frame, it may be determined whether a lumen is detected in the selected OCT image frame. If it is determined that no lumen has been detected in the OCT image frame, then the OCT image frame may be removed from further image processing and the process continues. Alternatively, if the lumen is detected in the frame, then a further determination of whether the detected lumen is affected by any artifact may be performed. If the detected lumen is affected by an artifact, then the OCT image frame may be removed from further processing and the process proceeds. If the detected lumen is not affected by any artifact, then it may be determined if the selected OCT image frame is the last OCT image frame from the OCT image. If the selected frame is not the last frame in the OCT image, then the next OCT image frame from the OCT image may be selected and the process returns to the lumen detection on the frame step. If the selected OCT image frame is the last OCT image frame, then the process proceeds. After removing the inappropriate OCT image frames, all the OCT image frames in which stent-struts are detected may be selected (Group Gₛ′). Then, a first OCT image frame from the selected OCT image frames in which stent-struts are detected may be selected. Subsequently, for the selected first OCT image frame, the distance between the lumen edge and stent-strut detected in first OCT image frame may be measured. Stent apposition may be evaluated. The stent apposition may be evaluated by comparing the measured distance between the lumen edge and stent-strut to the stent-strut width that is obtained from the stent information. The stent apposition value and an indicator for stent apposition level may be saved for the corresponding OCT image frame. Then, it may be determined whether the selected OCT image frame is the last OCT image frame, if the selected frame is the last frame, then the process ends. In this example the selected OCT image frame is the first OCT image frame, so a second OCT image frame is selected and the process returns to the aforementioned measure distance step. The process repeats until each OCT image frame selected is evaluated and a stent apposition value is obtained.

While GUI embodiments or displays of the present disclosure show the images or views vertically (see e.g., FIGS. 5A-5C), the orientation and location of the different imaging modalities or different views may be changed or modified in one or more embodiments as desired by a user. For example, an angiography image may be displayed on one side and an intravascular image may be displayed on another side of a display. In one or more other embodiments, the branch views may be shown to the side or on top of the carpet view.

In one or more embodiments, a GUI may display one or more values (e.g., lumen area, mean diameter, min. diameter, max. diameter, etc.). Such information may be used to determine or decide how to plan or proceed with a procedure, e.g., what stent size to use when the procedure relates to expansion/underexpansion or apposition/malapposition, to confirm that the stent used is of appropriate size and placement, etc.

As aforementioned, evaluating underexpansion/expansion and/or apposition/malapposition are examples of some of the applications of one or more embodiments of the present disclosure. One or more embodiments of the present disclosure may involve one or more additional or alternative applications, such as, but not limited to, determining whether plaque tissue, or a buildup of calcium, requires further attention. Another application example may involve identifying or determining diagnosis information, determining whether medical attention is needed or not, identifying a region of choice or interest, etc.

One or more embodiments of the present disclosure may include taking multiple views (e.g., OCT image, carpet view, ring view, tomo view, anatomical view, etc.), and one or more embodiments may highlight or emphasize NIRF/NIRAF. In one or more embodiments, two handles may operate as endpoints that may bound the color extremes of the NIRF/NIRAF data in or more embodiments. In one or more embodiments, the two handles may indicate a corresponding cut or area displayed in the 3D view.

In addition to the standard tomographic view, the user may select to display multiple longitudinal views. When connected to an angiography system, the Graphical User Interface (GUI) may also display angiography images.

In accordance with one or more aspects of the present disclosure, the aforementioned features are not limited to being displayed or controlled using any particular GUI. In general, the aforementioned imaging modalities may be used in various ways, including with or without one or more features of aforementioned embodiments of a GUI or GUIs. For example, a GUI may show an OCT image with a tool or marker to change the image view as aforementioned even if not presented with a GUI (or with one or more other components of a GUI; in one or more embodiments, the display may be simplified for a user to display set or desired information).

The procedure to select the region of interest and the position of a marker, an angle, a plane, etc., for example, using a touch screen, a GUI (or one or more components of a GUI; in one or more embodiments, the display may be simplified for a user to display the set or desired information), a processor (e.g., processor or computer 2, 1200, 1200′, or any other processor discussed herein) may involve, in one or more embodiments, a single press with a finger and dragging on the area to make the selection or modification. The new orientation and updates to the view may be calculated upon release of a finger, or a pointer.

For one or more embodiments using a touch screen, two simultaneous touch points may be used to make a selection or modification, and may update the view based on calculations upon release.

One or more functions may be controlled with one of the imaging modalities, such as the angiography image view or the OCT image view, to centralize user attention, maintain focus, and allow the user to see all relevant information in a single moment in time.

In one or more embodiments, one imaging modality may be displayed or multiple imaging modalities may be displayed.

One or more procedures may be used in one or more embodiments to select a region of choice or a region of interest for a view. For example, after a single touch is made on a selected area (e.g., by using a touch screen, by using a mouse or other input device to make a selection, etc.), a semi-circle (or other geometric shape used for the designated area) may automatically adjust to the selected region of choice or interest. Two (2) single touch points may operate to connect/draw the region of choice or interest. A single touch on a tomo or tomographic view (e.g., the OCT view 403 or 603) may operate to sweep around the tomo view, and may connect to form the region of choice or interest.

FIG. 8A shows an OCT system 100 (as referred to herein as “system 100” or “the system 100”) which may be used for one or more imaging modalities and/or for FFR calculation(s) in accordance with one or more aspects of the present disclosure. The system 100 comprises a light source 101, a reference arm 102, a sample arm 103, a deflected or deflecting section 108, a reference mirror (also referred to as a “reference reflection”, “reference reflector”, “partially reflecting mirror” and a “partial reflector”) 105, and one or more detectors 107 (which may be connected to a computer 1200). In one or more embodiments, the system 100 may include a patient interface device or unit (“PIU”) 110 and a catheter 120 (see e.g., embodiment examples of a PIU and a catheter as shown in FIG. 1A-1B, FIG. 3 and/or FIGS. 8A-8C), and the system 100 may interact with an object or sample 106, a patient (e.g., a blood vessel of a patient) 106, etc. (e.g., via the catheter 120 and/or the PIU 110). In one or more embodiments, the system 100 includes an interferometer or an interferometer is defined by one or more components of the system 100, such as, but not limited to, at least the light source 101, the reference arm 102, the sample arm 103, the deflecting section 108, and the reference mirror 105.

In accordance with one or more further aspects of the present disclosure, bench top systems may be utilized with multiple imaging modalities as disclosed herein. FIG. 8B shows an example of a system that can utilize the one or more imaging modalities and related methods discussed herein for a bench-top such as for ophthalmic applications and/or for FRR calculation(s). A light from a light source 101 delivers and splits into a reference arm 102 and a sample arm 103 with a deflecting section 108. A reference beam goes through a length adjustment section 904 and is reflected from a reference mirror (such as or similar to the reference mirror or reference reflection 105 shown in FIG. 8A) in the reference arm 102 while a sample beam is reflected or scattered from an object, a sample, a patient (e.g., blood vessel of a patient), etc. 106 in the sample arm 103 (e.g., via the PIU 110 and the catheter 120). In one embodiment, both beams combine at the deflecting section 108 and generate interference patterns. In one or more embodiments, the beams go to the combiner 903, and the combiner 903 combines both beams via the circulator 901 and the deflecting section 108, and the combined beams are delivered to one or more detectors (such as the one or more detectors 107). The output of the interferometer is continuously acquired with one or more detectors, such as the one or more detectors 107. The electrical analog signals are converted to the digital signals to analyze them with a computer, such as, but not limited to, the computer 1200 (see FIGS. 8A-8C; also shown in FIG. 10 discussed further below), the computer 1200′ (see e.g., FIG. 11 discussed further below), the computer 2 (see FIG. 1A), the image processor 50 or the computer 1200 (see FIG. 1B), any other computer or processor discussed herein, etc. Additionally or alternatively, one or more of the computers, CPUs, processors, etc. discussed herein may be used to process, control, update, emphasize, and/or change one or more of imaging modalities, and/or process the related techniques, functions or methods, or may process the electrical signals as discussed above.

The electrical analog signals may be converted to the digital signals to analyze the digital signals with a computer, such as, but not limited to, the computer 1200 (see FIGS. 1B and 8A-8C; also shown in FIG. 10 discussed further below), the computer 1200′ (see e.g., FIG. 11 discussed further below), the computer 2 (see FIG. 1A), the image processor 50 or the computer 1200 (see FIG. 1B), any other processor or computer discussed herein, etc. Additionally or alternatively, one or more of the computers, CPUs, processors, etc. discussed herein may be used to process, control, update, emphasize, and/or change one or more imaging modalities, and/or process the related techniques, functions or methods, or may process the electrical signals as discussed above. In one or more embodiments (see e.g., FIG. 8B), the sample arm 103 includes the PIU 110 and the catheter 120 so that the sample beam is reflected or scattered from the object, patient (e.g., blood vessel of a patient), etc. 106 as discussed herein. In one or more embodiments, the PIU 110 may include one or more motors to control the pullback operation of the catheter 120 (or one or more components thereof) and/or to control the rotation or spin of the catheter 120 (or one or more components thereof) (see e.g., the motor M of FIG. 1B). For example, as best seen in FIG. 8B, the PIU 110 may include a pullback motor (PM) and a spin motor (SM), and/or may include a motion control unit 112 that operates to perform the pullback and/or rotation features using the pullback motor PM and/or the spin motor SM. As discussed herein, the PIU 110 may include a rotary junction (e.g., rotary junction RJ as shown in FIGS. 8B and 8C). The rotary junction RJ may be connected to the spin motor SM so that the catheter 120 may obtain one or more views or images of the object, sample, patient (e.g., blood vessel of a patient), etc. 106. The computer 1200 (or the computer 1200′, computer 2, any other computer or processor discussed herein, etc.) may be used to control one or more of the pullback motor PM, the spin motor SM and/or the motion control unit 112. An OCT system may include one or more of a computer (e.g., the computer 1200, the computer 1200′, computer 2, any other computer or processor discussed herein, etc.), the PIU 110, the catheter 120, a monitor (such as the display 1209), etc. One or more embodiments of an OCT system may interact with one or more external systems, such as, but not limited to, an angio system, external displays, one or more hospital networks, external storage media, a power supply, a bedside controller (e.g., which may be connected to the OCT system (or other intravascular imaging modality system) using Bluetooth technology or other methods known for wireless communication), etc.

In one or more embodiments including the deflecting or deflected section 108 (best seen in FIGS. 8A-8C), the deflected section 108 may operate to deflect the light from the light source 101 to the reference arm 102 and/or the sample arm 103, and then send light received from the reference arm 102 and/or the sample arm 103 towards the at least one detector 107 (e.g., a spectrometer, one or more components of the spectrometer, another type of detector, etc.). In one or more embodiments, the deflected section (e.g., the deflected section 108 of the system 100, 100′, 100″, any other system discussed herein, etc.) may include or may comprise one or more interferometers or optical interference systems that operate as described herein, including, but not limited to, a circulator, a beam splitter, an isolator, a coupler (e.g., fusion fiber coupler), a partially severed mirror with holes therein, a partially severed mirror with a tap, etc. In one or more embodiments, the interferometer or the optical interference system may include one or more components of the system 100 (or any other system discussed herein) such as, but not limited to, one or more of the light source 101, the deflected section 108, the rotary junction RJ, a PIU 110, a catheter 120, etc. One or more features of the aforementioned configurations of at least FIGS. 1A-11 may be incorporated into one or more of the systems, including, but not limited to, the system 100, 100′, 100″, discussed herein.

In accordance with one or more further aspects of the present disclosure, one or more other systems may be utilized with one or more of the multiple imaging modalities and related method(s) as disclosed herein. FIG. 8C shows an example of a system 100″ that may utilize the one or more multiple imaging modalities and/or related technique(s) or method(s) such as for ophthalmic applications and/or for FFR calculation(s). FIG. 8C shows an exemplary schematic of an OCT-fluorescence imaging system 100″, according to one or more embodiments of the present disclosure. An OCT light source 101 (e.g., with a 1.3 μm) is delivered and split into a reference arm 102 and a sample arm 103 with a deflector or deflected section (e.g., a splitter) 108, creating a reference beam and sample beam, respectively. The reference beam from the OCT light source 101 is reflected by a reference mirror 105 while a sample beam is reflected or scattered from an object (e.g., an object to be examined, an object, a patient, etc.) 106 through a circulator 901, a rotary junction 90 (“RJ”) and a catheter 120. In one or more embodiments, the fiber between the circulator 901 and the reference mirror or reference reflection 105 may be coiled to adjust the length of the reference arm 102 (best seen in FIG. 8C). Optical fibers in the sample arm 103 may be made of double clad fiber (“DCF”). Excitation light for the fluorescence may be directed to the RJ 90 and the catheter 120, and illuminate the object (e.g., an object to be examined, an object, a patient, etc.) 106. The light from the OCT light source 101 may be delivered through the core of DCF while the fluorescence light emitted from the object (e.g., an object to be examined, an object, a patient, etc.) 106 may be collected through the cladding of the DCF. For pullback imaging, the RJ 90 may be moved with a linear stage to achieve helical scanning of the object (e.g., an object to be examined, an object, a patient, etc.) 106. In one or more embodiments, the RJ 90 may include any one or more features of an RJ as discussed herein. Dichroic filters DF1, DF2 may be used to separate excitation light and the rest of fluorescence and OCT lights. For example (and while not limited to this example), in one or more embodiments, DF1 may be a long pass dichroic filter with a cutoff wavelength of ˜1000 nm, and the OCT light, which may be longer than a cutoff wavelength of DF1, may go through the DF1 while fluorescence excitation and emission, which are a shorter wavelength than the cut off, reflect at DF1. In one or more embodiments, for example (and while not limited to this example), DF2 may be a short pass dichroic filter; the excitation wavelength may be shorter than fluorescence emission light such that the excitation light, which has a wavelength shorter than a cutoff wavelength of DF2, may pass through the DF2, and the fluorescence emission light reflect with DF2. In one embodiment, both beams combine at the deflecting section 108 and generate interference patterns. In one or more embodiments, the beams go to the coupler or combiner 903, and the coupler or combiner 903 combines both beams via the circulator 901 and the deflecting section 108, and the combined beams are delivered to one or more detectors (such as the one or more detectors 107; see e.g., the first detector 107 connected to the coupler or combiner 903 in FIG. 8C).

In one or more embodiments, the optical fiber in the catheter 120 operates to rotate inside the catheter 120, and the OCT light and excitation light may be emitted from a side angle of a tip of the catheter 120. After interacting with the object or patient 106, the OCT light may be delivered back to an OCT interferometer (e.g., via the circulator 901 of the sample arm 103), which may include the coupler or combiner 903, and combined with the reference beam (e.g., via the coupler or combiner 903) to generate interference patterns. The output of the interferometer is detected with a first detector 107, wherein the first detector 107 may be photodiodes or multi-array cameras, and then may be recorded to a computer (e.g., to the computer 2, the computer 1200 as shown in FIG. 8C, the computer 1200′, or any other computer discussed herein) through a first data-acquisition unit or board (“DAQ1”).

Simultaneously or at a different time, the fluorescence intensity may be recorded through a second detector 107 (e.g., a photomultiplier) through a second data-acquisition unit or board (“DAQ2”). The OCT signal and fluorescence signal may be then processed by the computer (e.g., to the computer 2, the computer 1200 as shown in FIG. 8C, the computer 1200′, or any other computer discussed herein) to generate an OCT-fluorescence dataset 140, which includes or is made of multiple frames of helically scanned data. Each set of frames includes or is made of multiple data elements of co-registered OCT and fluorescence data, which correspond to the rotational angle and pullback position.

Detected fluorescence or auto-fluorescence signals may be processed or further processed as discussed in U.S. Pat. App. No. 62/861,888, filed on Jun. 14, 2019, the disclosure of which is incorporated herein by reference in its entirety, and/or as discussed in U.S. patent application Ser. No. 16/368,510, filed Mar. 28, 2019, and published as U.S. Pat. Pub. No. 2019/0298174 on Oct. 3, 2019, the disclosure of which is incorporated herein by reference herein in its entirety.

While not limited to such arrangements, configurations, devices or systems, one or more embodiments of the devices, apparatuses, systems, methods, storage mediums, GUI's, etc. discussed herein may be used with an apparatus or system as aforementioned, such as, but not limited to, for example, the system 100, the system 100′, the system 100″, the devices, apparatuses, or systems of FIGS. 1A-11, any other device, apparatus or system discussed herein, etc. In one or more embodiments, one user may perform the method(s) discussed herein. In one or more embodiments, one or more users may perform the method(s) discussed herein. In one or more embodiments, one or more of the computers, CPUs, processors, etc. discussed herein may be used to process, control, update, emphasize, and/or change one or more of the imaging modalities, and/or process the related techniques, functions or methods, or may process the electrical signals as discussed above.

The light source 101 may include a plurality of light sources or may be a single light source. The light source 101 may be a broadband lightsource, and may include one or more of a laser, an organic light emitting diode (OLED), a light emitting diode (LED), a halogen lamp, an incandescent lamp, supercontinuum light source pumped by a laser, and/or a fluorescent lamp. The light source 101 may be any light source that provides light which may then be dispersed to provide light which is then used for imaging, performing control, viewing, changing, emphasizing methods for imaging modalities, constructing or reconstructing 2D or 3D structure(s), performing FFR calculation(s), and/or any other method discussed herein. The light source 101 may be fiber coupled or may be free space coupled to the other components of the apparatus and/or system 100, 100′, 100″, the devices, apparatuses or systems of FIGS. 1A-11, or any other embodiment discussed herein. As aforementioned, the light source 101 may be a swept-source (SS) light source.

Additionally or alternatively, the one or more detectors 107 may be a linear array, a charge-coupled device (CCD), a plurality of photodiodes or some other method of converting the light into an electrical signal. The detector(s) 107 may include an analog to digital converter (ADC). The one or more detectors may be detectors having structure as shown in one or more of FIGS. 1A-11 and as discussed above.

In accordance with one or more aspects of the present disclosure, one or more methods for performing imaging are provided herein. FIG. 9 illustrates a flow chart of at least one embodiment of a method for performing imaging. The method(s) may include one or more of the following: (i) splitting or dividing light into a first light and a second reference light (see step S4000 in FIG. 9); (ii) receiving reflected or scattered light of the first light after the first light travels along a sample arm and irradiates an object (see step S4001 in FIG. 9); (iii) receiving the second reference light after the second reference light travels along a reference arm and reflects off of a reference reflection (see step S4002 in FIG. 9); and (iv) generating interference light by causing the reflected or scattered light of the first light and the reflected second reference light to interfere with each other (for example, by combining or recombining and then interfering, by interfering, etc.), the interference light generating one or more interference patterns (see step S4003 in FIG. 9). One or more methods may further include using low frequency monitors to update or control high frequency content to improve image quality. For example, one or more embodiments may use multiple imaging modalities, related methods or techniques for same, etc. to achieve improved image quality. In one or more embodiments, an imaging probe may be connected to one or more systems (e.g., the system 100, the system 100′, the system 100″, the devices, apparatuses or systems of FIGS. 1A-11, any other system or apparatus discussed herein, etc.) with a connection member or interface module. For example, when the connection member or interface module is a rotary junction for an imaging probe, the rotary junction may be at least one of: a contact rotary junction, a lenseless rotary junction, a lens-based rotary junction, or other rotary junction known to those skilled in the art. The rotary junction may be a one channel rotary junction or a two channel rotary junction. In one or more embodiments, the illumination portion of the imaging probe may be separate from the detection portion of the imaging probe. For example, in one or more applications, a probe may refer to the illumination assembly, which includes an illumination fiber (e.g., single mode fiber, a GRIN lens, a spacer and the grating on the polished surface of the spacer, etc.). In one or more embodiments, a scope may refer to the illumination portion which, for example, may be enclosed and protected by a drive cable, a sheath, and detection fibers (e.g., multimode fibers (MMFs)) around the sheath. Grating coverage is optional on the detection fibers (e.g., MMFs) for one or more applications. The illumination portion may be connected to a rotary joint and may be rotating continuously at video rate. In one or more embodiments, the detection portion may include one or more of: a detection fiber, a detector (e.g., the one or more detectors 107, a spectrometer, etc.), the computer 1200, the computer 1200′, the computer 2, any other computer or processor discussed herein, etc. The detection fibers may surround the illumination fiber, and the detection fibers may or may not be covered by a grating, a spacer, a lens, an end of a probe or catheter, etc.

The one or more detectors 107 may transmit the digital or analog signals to a processor or a computer such as, but not limited to, an image processor, a processor or computer 1200, 1200′ (see e.g., 8A-8C and 10-11), a computer 2 (see e.g., FIG. 1A), a processor or computer 1200 or an image processor 50 (see e.g., FIG. 1B), any other processor or computer discussed herein, a combination thereof, etc. The image processor may be a dedicated image processor or a general purpose processor that is configured to process images. In at least one embodiment, the computer 1200, 1200′, 2 or any other processor or computer discussed herein may be used in place of, or in addition to, the image processor or any other processor discussed herein. In an alternative embodiment, the image processor may include an ADC and receive analog signals from the one or more detectors 107. The image processor may include one or more of a CPU, DSP, FPGA, ASIC, or some other processing circuitry. The image processor may include memory for storing image, data, and instructions. The image processor may generate one or more images based on the information provided by the one or more detectors 107. A computer or processor discussed herein, such as, but not limited to, a processor of the devices, apparatuses or systems of FIGS. 1A-8C, the computer 1200, the computer 1200′, the computer 2, the image processor, may also include one or more components further discussed herein below (see e.g., FIGS. 10-11).

In at least one embodiment, a console or computer 1200, 1200′, a computer 2, any other computer or processor discussed herein, etc. operates to control motions of the RJ via the motion control unit (MCU) 112 or a motor M, acquires intensity data from the detector(s) in the one or more detectors 107, and displays the scanned image (e.g., on a monitor or screen such as a display, screen or monitor 1209 as shown in the console or computer 1200 of any of FIGS. 8A-8C and FIG. 10 and/or the console 1200′ of FIG. 11 as further discussed below; the computer 2 of FIG. 1A; any other computer or processor discussed herein; etc.). In one or more embodiments, the MCU 112 or the motor M operates to change a speed of a motor of the RJ and/or of the RJ. The motor may be a stepping or a DC servo motor to control the speed and increase position accuracy (e.g., compared to when not using a motor, compared to when not using an automated or controlled speed and/or position change device, compared to a manual control, etc.).

The output of the one or more components of any of the systems discussed herein may be acquired with the at least one detector 107, e.g., such as, but not limited to, photodiodes, Photomultiplier tube(s) (PMTs), line scan camera(s), or multi-array camera(s). Electrical analog signals obtained from the output of the system 100, 100′, 100″, and/or the detector(s) 107 thereof, and/or from the devices, apparatuses, or systems of FIGS. 1A-8C, are converted to digital signals to be analyzed with a computer, such as, but not limited to, the computer 1200, 1200′. In one or more embodiments, the light source 101 may be a radiation source or a broadband light source that radiates in a broad band of wavelengths. In one or more embodiments, a Fourier analyzer including software and electronics may be used to convert the electrical analog signals into an optical spectrum.

Unless otherwise discussed herein, like numerals indicate like elements. For example, while variations or differences exist between the systems, such as, but not limited to, the system 100, the system 100′, the system 100″, or any other device, apparatus or system discussed herein, one or more features thereof may be the same or similar to each other, such as, but not limited to, the light source 101 or other component(s) thereof (e.g., the console 1200, the console 1200′, etc.). Those skilled in the art will appreciate that the light source 101, the motor or MCU 112, the RJ, the at least one detector 107, and/or one or more other elements of the system 100 may operate in the same or similar fashion to those like-numbered elements of one or more other systems, such as, but not limited to, the devices, apparatuses or systems of FIGS. 1A-8C, the system 100′, the system 100″, or any other system discussed herein. Those skilled in the art will appreciate that alternative embodiments of the devices, apparatuses or systems of FIGS. 1A-8C, the system 100′, the system 100″, any other device, apparatus or system discussed herein, etc., and/or one or more like-numbered elements of one of such systems, while having other variations as discussed herein, may operate in the same or similar fashion to the like-numbered elements of any of the other systems (or components thereof) discussed herein. Indeed, while certain differences exist between the system 100 of FIG. 8A and one or more embodiments shown in any of FIGS. 1A-7 and 8B-8C, for example, as discussed herein, there are similarities. Likewise, while the console or computer 1200 may be used in one or more systems (e.g., the system 100, the system 100′, the system 100″, the devices, apparatuses or systems of any of FIGS. 1A-11, or any other system discussed herein, etc.), one or more other consoles or computers, such as the console or computer 1200′, any other computer or processor discussed herein, etc., may be used additionally or alternatively.

There are many ways to compute intensity, viscosity, resolution (including increasing resolution of one or more images), etc., to use one or more imaging modalities, to calculate FFR, and/or related methods for same, discussed herein, digital as well as analog. In at least one embodiment, a computer, such as the console or computer 1200, 1200′, may be dedicated to control and monitor the imaging (e.g., OCT, single mode OCT, multimodal OCT, multiple imaging modalities, etc.) devices, systems, methods and/or storage mediums described herein.

The electric signals used for imaging may be sent to one or more processors, such as, but not limited to, a computer or processor 2 (see e.g., FIG. 1A), a computer 1200 (see e.g., FIGS. 8A-8C and 10), a computer 1200′ (see e.g., FIG. 11), etc. as discussed further below, via cable(s) or wire(s), such as, but not limited to, the cable(s) or wire(s) 113 (see FIG. 10). Additionally or alternatively, the electric signals, as aforementioned, may be processed in one or more embodiments as discussed above by any other computer or processor or components thereof. The computer or processor 2 as shown in FIG. 1A may be used instead of any other computer or processor discussed herein (e.g., computer or processors 1200, 1200′, etc.), and/or the computer or processor 1200, 1200′ may be used instead of any other computer or processor discussed herein (e.g., computer or processor 2). In other words, the computers or processors discussed herein are interchangeable, and may operate to perform any of the one or more imaging modalities feature(s) and method(s) discussed herein, including using, controlling, and changing a GUI or multiple GUI's and/or calculating FFR.

Various components of a computer system 1200 are provided in FIG. 10. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., including but not limited to, being connected to the console, the probe, the imaging apparatus or system, any motor discussed herein, a light source, etc.). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a device or system, such as, but not limited to, an apparatus or system using one or more imaging modalities and related method(s) as discussed herein), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The system 1200 may include one or more additional processors in addition to CPU 1201, and such processors, including the CPU 1201, may be used for tissue or object characterization, diagnosis, evaluation, imaging and/or construction or reconstruction, as well as FFR calculation(s). The system 1200 may further include one or more processors connected via a network connection (e.g., via network 1206). The CPU 1201 and any additional processor being used by the system 1200 may be located in the same telecom network or in different telecom networks (e.g., performing feature(s), function(s), technique(s), method(s), etc. discussed herein may be controlled remotely).

The I/O or communication interface 1205 provides communication interfaces to input and output devices, which may include a light source, a spectrometer, a microphone, a communication cable and a network (either wired or wireless), a keyboard 1210, a mouse (see e.g., the mouse 1211 as shown in FIG. 11), a touch screen or screen 1209, a light pen and so on. The communication interface of the computer 1200 may connect to other components discussed herein via line 113 (as diagrammatically shown in FIG. 10). The Monitor interface or screen 1209 provides communication interfaces thereto.

Any methods and/or data of the present disclosure, such as the methods for performing tissue or object characterization, diagnosis, examination, imaging (including, but not limited to, increasing image resolution, performing imaging using one or more imaging modalities, viewing or changing one or more imaging modalities and related methods (and/or option(s) or feature(s)), etc.), and/or FFR calculation(s), for example, as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”) a digital versatile disc (“DVD”), a Blu-Ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 11), SRAM, etc.), an optional combination thereof, a server/database, etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) of the present disclosure may also be realized by a computer of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, systems, and computer-readable storage mediums related to the processors, such as, but not limited to, the processor of the aforementioned computer 1200, etc., as described above may be achieved utilizing suitable hardware, such as that illustrated in the figures. Functionality of one or more aspects of the present disclosure may be achieved utilizing suitable hardware, such as that illustrated in FIG. 10. Such hardware may be implemented utilizing any of the known technologies, such as standard digital circuitry, any of the known processors that are operable to execute software and/or firmware programs, one or more programmable digital devices or systems, such as programmable read only memories (PROMs), programmable array logic devices (PALs), etc. The CPU 1201 (as shown in FIG. 10), the processor or computer 2 (as shown in FIG. 1A) and/or the computer or processor 1200′ (as shown in FIG. 11) may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 2, 1200, 1200′, etc.) may include the aforementioned CPU structure, or may be connected to such CPU structure for communication therewith.

As aforementioned, hardware structure of an alternative embodiment of a computer or console 1200′ is shown in FIG. 11. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid state drive (SSD) 1207. The computer or console 1200′ may include a display 1209. The computer 1200′ may connect with a motor, a console, or any other component of the device(s) or system(s) discussed herein via the operation interface 1214 or the network interface 1212 (e.g., via a cable or fiber, such as the cable or fiber 113 as similarly shown in FIG. 10). A computer, such as the computer 1200′, may include a motor or motion control unit (MCU) in one or more embodiments. The operation interface 1214 is connected with an operation unit such as a mouse device 1211, a keyboard 1210 or a touch panel device. The computer 1200′ may include two or more of each component.

At least one computer program is stored in the SSD 1207, and the CPU 1201 loads the at least one program onto the RAM 1203, and executes the instructions in the at least one program to perform one or more processes described herein, as well as the basic input, output, calculation, memory writing and memory reading processes.

The computer, such as the computer 2, the computer 1200, 1200′, (or other component(s) such as, but not limited to, the PCU, etc.), etc. may communicate with an MCU, an interferometer, a spectrometer, a detector, etc. to perform imaging, and reconstructs an image from the acquired intensity data. The monitor or display 1209 displays the reconstructed image, and may display other information about the imaging condition or about an object to be imaged. The monitor 1209 also provides a graphical user interface for a user to operate any system discussed herein. An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs any system discussed herein to set or change the imaging condition (e.g., improving resolution of an image or images), and to start or end the imaging. A light or laser source and a spectrometer and/or detector may have interfaces to communicate with the computers 1200, 1200′ to send and receive the status information and the control signals.

While not limited to such arrangements, configurations, devices or systems, one or more embodiments of the devices, apparatuses, systems, methods, storage mediums, etc. discussed herein may be used with an apparatus or system as aforementioned, such as, but not limited to, for example, the system 10, the system 20, the system 100, the system 100′, the system 100″, the devices, apparatuses, or systems of FIGS. 1A-11, any other device, apparatus or system discussed herein, etc. In one or more embodiments, one user may perform the method(s) discussed herein. In one or more embodiments, one or more users may perform the method(s) discussed herein. In one or more embodiments, one or more of the computers, CPUs, processors, etc. discussed herein may be used to process, control, update, emphasize, and/or change one or more of the imaging modalities, and/or process the related techniques, functions or methods, such as, but not limited to, FFR calculation(s), or may process the electrical signals as discussed above.

The present disclosure and/or one or more components of devices, systems and storage mediums, and/or methods, thereof also may be used in conjunction with optical coherence tomography probes. Such probes include, but are not limited to, the OCT imaging systems disclosed in U.S. Pat. Nos. 6,763,261; 7,366,376; 7,843,572; 7,872,759; 8,289,522; 8,676,013; 8,928,889; 9,087,368; 9,557,154; 10,912,462; 9,795,301; and U.S. Pat. No. 9,332,942 to Tearney et al. and arrangements and methods of facilitating photoluminescence imaging, such as those disclosed in U.S. Pat. No. 7,889,348 to Tearney et al., as well as the disclosures directed to multimodality imaging disclosed in U.S. Pat. No. 9,332,942 and U.S. Patent Publication Nos. 2010/0092389, 2011/0292400, 2012/0101374, 2014/0276011, 2017/0135584, 2016/0228097, 2018/0045501 and 2018/0003481, WO 2016/015052 to Tearney et al. and WO 2016/144878, each of which patents, patent publications and patent application(s) are incorporated by reference herein in their entireties. As aforementioned, any feature or aspect of the present disclosure may be used with OCT imaging systems, apparatuses, methods, storage mediums or other aspects or features as discussed in U.S. patent application Ser. No. 16/414,222, filed on May 16, 2019, the entire disclosure of which is incorporated by reference herein in its entirety, as discussed in U.S. Pat. Pub. No. 2019/0374109, which was published on Dec. 12, 2019, the disclosure of which is incorporated by reference herein in its entirety, as discussed in U.S. Pat. App. No. 62/944,064, filed on Dec. 5, 2019, the disclosure of which is incorporated by reference herein in its entirety, as discussed in U.S. Pat. Pub. No. 2021/0077037, published on Mar. 18, 2021, as discussed in U.S. Pat. Pub. No. 2021/0174125, published on Jun. 10, 2021, and as discussed in U.S. patent application Ser. No. 17/098,042, filed on Nov. 13, 2020, the disclosure of which is incorporated by reference herein in its entirety.

The present disclosure and/or one or more components of devices, systems, and storage mediums, and/or methods, thereof also may be used in conjunction with OCT imaging systems and/or catheters and catheter systems, such as, but not limited to, those disclosed in U.S. Pat. Nos. 9,869,828; 10,323,926; 10,558,001; 10,601,173; 10,606,064; 10,743,749; 10,884,199; 10,895,692; and 11,175,126 as well as U.S. Patent Publication Nos. 2019/0254506; 2020/0390323; 2021/0121132; 2021/0174125; 2022/0040454; 2022/0044428; and WO2021/055837, each of which patents and patent publications are incorporated by reference herein in their entireties.

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Provisional Pat. App. No. 63/150,859, filed on Feb. 18, 2021, the disclosure of which is incorporated by reference herein in its entirety. Such endoscope devices, systems, methods, and/or storage mediums are disclosed in at least: U.S. patent application Ser. No. 17/565,319, filed on Dec. 29, 2021, the disclosure of which is incorporated by reference herein in its entirety; U.S. Pat. App. No. 63/132,320, filed on Dec. 30, 2020, the disclosure of which is incorporated by reference herein in its entirety; U.S. patent application Ser. No. 17/564,534, filed on Dec. 29, 2021, the disclosure of which is incorporated by reference herein in its entirety; and U.S. Pat. App. No. 63/131,485, filed Dec. 29, 2020, the disclosure of which is incorporated by reference herein in its entirety. Such continuum robotic systems and catheters may also include, but are not limited to, those described in U.S. Patent Publication Nos. 2019/0105468; 2021/0369085; 2020/0375682; 2021/0121162; 2021/0121051; and 2022-0040450, each of which patents and/or patent publications are incorporated by reference herein in their entireties.

References [1]-[26] that may be cited above, and that are incorporated by reference herein in their entireties are, as follows:
[1] P. A. L. Tonino et al., “Fractional Flow Reserve versus Angiography for Guiding Percutaneous Coronary Intervention,” N. Engl. J. Med., vol. 360, no. 3, pp. 213-224, 2009, doi: Doi 10.1056/Nejmoao807611;
[2] S. J. Park et al., “Visual-Functional Mismatch Between Coronary Angiography and Fractional Flow Reserve,” JACC Cardiovasc. Interv., vol. 5, no. 10, pp. 1029-1036 October 2012, doi: 10.1016/J.JCIN.2012.07.007;
[3] B. De Bruyne et al., “Fractional Flow Reserve-Guided PCI versus Medical Therapy in Stable Coronary Disease,” N. Engl. J. Med., vol. 367, no. 11, pp. 991-1001 September 2012, doi: 10.1056/NEJMoa1205361;
[4] P. Xaplanteris et al., “Five-Year Outcomes with PCI Guided by Fractional Flow Reserve,” N. Engl. J. Med., vol. 379, no. 3, pp. 250-259, July 2018, doi: 10.1056/NEJMOA1803538/SUPPL_FILE/NEJMOA1803538_DISCLOSURES.PDF;
[5] C. E. Coles et al., “Partial-breast radiotherapy after breast conservation surgery for patients with early breast cancer (UK IMPORT LOW trial): 5-year results from a multicentre, randomised, controlled, phase 3, non-inferiority trial,” Lancet, vol. 390, no. 10099, pp. 1048-1060 September 2017, doi: 10.1016/S0140-6736(17)31145-5;
[6] B. K. Koo et al., “Diagnosis of ischemia-causing coronary stenoses by noninvasive fractional flow reserve computed from coronary computed tomographic angiograms: Results from the prospective multicenter DISCOVER-FLOW (Diagnosis of Ischemia-Causing Stenoses Obtained Via Noninvasive Fractional Flow Reserve) study,” J. Am. Coll. Cardiol., vol. 58, no. 19, pp. 1989-1997 November 2011, doi: 10.1016/J.JACC.2011.06.066;
[7] J. K. Min et al., “Diagnostic Accuracy of Fractional Flow Reserve From Anatomic CT Angiography,” J. Am. Coll. Cardiol., vol. 63, no. 12, pp. 1145-1155 April 2014, Accessed: Mar. 5, 2019. [Online]. Available: http://www.ncbi.nlm.nih.gov/pubmed/24486266;
[8] R. Kornowski et al., “Fractional Flow Reserve Derived From Routine Coronary Angiograms,” J. Am. Coll. Cardiol., vol. 68, no. 20, pp. 2235-2237 November 2016, doi: 10.1016/J.JACC.2016.08.051;
[9] G. Witberg et al., “Diagnostic Performance of Angiogram-Derived Fractional Flow Reserve: A Pooled Analysis of 5 Prospective Cohort Studies,” JACC Cardiovasc. Interv., vol. 13, no. 4, pp. 488-497, February 2020, doi: 10.1016/J.JCIN.2019.10.045;
[10] J. Westra et al., “Diagnostic Performance of In-Procedure Angiography-Derived Quantitative Flow Reserve Compared to Pressure-Derived Fractional Flow Reserve: The FAVOR II Europe-Japan Study,” doi: 10.1161/JAHA.118.009603;
[11] K. Masdjedi et al., “Validation of a three-dimensional quantitative coronary angiography-based software to calculate fractional flow reserve: The FAST study,” EuroIntervention, vol. 16, no. 7, pp. 591-599, 2021, doi: 10.4244/EIJ-D-19-00466;
[12] C. A. Taylor, T. A. Fonte, and J. K. Min, “Computational Fluid Dynamics Applied to Cardiac Computed Tomography for Noninvasive Quantification of Fractional Flow Reserve: Scientific Basis,” J. Am. Coll. Cardiol., vol. 61, no. 22, pp. 2233-2241, June 2013, doi: 10.1016/J.JACC.2012.11.083;
[13] N. P. Johnson, R. L. Kirkeeide, and K. L. Gould, “Coronary anatomy to predict physiology fundamental limits,” Circ. Cardiovasc. Imaging, vol. 6, no. 5, pp. 817-832, September 2013, doi: 10.1161/CIRCIMAGING.113.000373;
[14] C. G. Bezerra et al., “Coronary fractional flow reserve derived from intravascular ultrasound imaging: Validation of a new computational method of fusion between anatomy and physiology,” Catheter. Cardiovasc. Interv., vol. 93, no. 2, pp. 266-274, February 2019, doi: 10.1002/CCD.27822;
[15] W. Yu et al., “Circulation: Cardiovascular Interventions Accuracy of Intravascular Ultrasound-Based Fractional Flow Reserve in Identifying Hemodynamic Significance of Coronary Stenosis,” Circ Cardiovasc Interv, vol. 14, p. 9840, 2021, doi: 10.1161/CIRCINTERVENTIONS.120.009840;
[16] W. Yu et al., “Diagnostic accuracy of intracoronary optical coherence tomography-derived fractional flow reserve for assessment of coronary stenosis severity,” EuroIntervention, vol. 15, no. 2, pp. 189-197, June 2019, doi: 10.4244/EIJ-D-19-00182;
[17] K. E. Lee, S. H. Lee, E.-S. Shin, and E. B. Shim, “A vessel length-based method to compute coronary fractional flow reserve from optical coherence tomography images,” Biomed. Eng. Online, doi: 10.1186/s12938-017-0365-4;
[18] F. Seike et al., “Intravascular Ultrasound-Derived Virtual Fractional Flow Reserve for the Assessment of Myocardial Ischemia,” Circ. J., vol. 82, no. 3, pp. 815-823, 2018, doi: 10.1253/CIRCJ.CJ-17-1042;
[19] F. Seike et al., “Intracoronary Optical Coherence Tomography-Derived Virtual Fractional Flow Reserve for the Assessment of Coronary Artery Disease,” Am. J. Cardiol., vol. 120, no. 10, pp. 1772-1779 November 2017, doi: 10.1016/J.AMJCARD.2017.07.083;
[20] R. L. Kirkeeide, K. L. Gould, and L. Parsel, “Assessment of coronary stenoses by myocardial perfusion imaging during pharmacologic coronary vasodilation. VII. Validation of coronary flow reserve as a single integrated functional measure of stenosis severity reflecting all its geometric dimensions,” J. Am. Coll. Cardiol., vol. 7, no. 1, pp. 103-113, January 1986, doi: 10.1016/S0735-1097 (86) 80266-2;
[21] K. L. Gould, K. O. Kelley, and E. L. Bolson, “Experimental validation of quantitative coronary arteriography for determining pressure-flow characteristics of coronary stenosis,” Circulation, vol. 66, no. 5 I, pp. 930-937, 1982, doi: 10.1161/01.CIR.66.5.930;
[22] B. De Bruyne et al., “Fractional Flow Reserve—Guided PCI for Stable Coronary Artery Disease,” N. Engl. J. Med., vol. 371, no. 13, pp. 1208-1217 September 2014, doi: 10.1056/NEJMOA1408758/SUPPL_FILE/NEJMOA1408758_DISCLOSURES.PDF;
[23] A. Jeremias et al., “Effects of Intravenous and Intracoronary Adenosine 5′-Triphosphate as Compared With Adenosine on Coronary Flow and Pressure Dynamics,” Circulation, vol. 101, no. 3, pp. 318-323, January 2000, doi: 10.1161/01.CIR.101.3.318;
[24] J. A. Leppo, “Comparison of pharmacologic stress agents,” J Nucl Cardiol., November-December 1996, 3 (6 Pt 2): S22-6, doi: 10.1016/s1071-3581(96)90204-4;
[25] H. Wieneke et al., “Corrected coronary flow velocity reserve: a new concept for assessing coronary perfusion,” J. Am. Coll. Cardiol., vol. 35, no. 7, pp. 1713-1720, June 2000, doi: 10.1016/S0735-1097 (00) 00639-2; and
[26] M. Miyagawa et al., “Thallium-201 myocardial tomography with intravenous infusion of adenosine triphosphate in diagnosis of coronary artery disease,” J. Am. Coll. Cardiol., vol. 26, no. 5, pp. 1196-1201 November 1995, doi: 10.1016/0735-1097(95)00304-5.

Although the disclosure herein has been described with reference to particular embodiments, it is to be understood that these embodiments are merely illustrative of the principles and applications of the present disclosure (and are not limited thereto), and the invention is not limited to the disclosed embodiments. It is therefore to be understood that numerous modifications may be made to the illustrative embodiments and that other arrangements may be devised without departing from the spirit and scope of the present disclosure. The scope of the following claims is to be accorded the broadest interpretation so as to encompass all such modifications and equivalent structures and functions.

1. An image processing apparatus comprising:
one or more processors that operate to:
obtain one or more intravascular images of one or more imaging modalities of an object or target during a pullback of a probe or catheter;
detect one or more arterial branches in the one or more intravascular images;
calculate or determine a pressure loss or change of the one or more arterial branches detected in the one or more intravascular images;
automatically calculate one or more Fractional Flow Reserve (FFR) values using the one or more intravascular images and using the calculated or determined pressure loss or change of the one or more arterial branches;
automatically calculate one or more areas of the detected one or more arterial branches; and
provide or display information to determine or assist in determining whether a stenosis and/or another medical condition exists in or overlaps with one or more of the calculated one or more areas of the detected one or more arterial branches based on the calculated one or more FFR values.

2. The image processing apparatus of claim 1, wherein the one or more processors further operate to one or more of the following:
detect lumen area(s) using a lumen detection method or technique;
detect a minimum lumen area (As) and define a stenotic area (L);
construct a carpet view of the pullback;
in a case where an arterial branch of the detected one or more arterial branches is within or has a portion that passes through or is within, or the calculated area or areas of the arterial branch is within or overlaps with, the stenotic area, reduce a velocity of a fluid or other object passing through the branch or lumen;
calculate a diastolic and systolic Stenotic Flow Reserve(s) (SFR) using the velocity, the stenotic area (L), and the minimum lumen area (As); and/or
use the SFR to calculate the Fractional Flow Reserve (FFR).

3. (canceled)

4. The image processing apparatus of claim 1, wherein the one or more processors further operate to detect a stenotic area in the one or more intravascular images and to calculate the FFR values for the stenotic area only where the pressure loss or change of the one or more arterial branches is occurring.

5. The image processing apparatus of claim 1, wherein the object or target is an organ, a tissue, a sample, a portion of a patient, a vessel, a blood vessel, or a patient.

6. The image processing apparatus of claim 1, wherein the one or more processors further operate to:
determine whether a Percutaneous Coronary Intervention (PCI) is needed for the object or target;
in a case where it is determined that the object or target needs the PCI, perform the PCI, or, in a case where it is determined that the object or target does not need the PCI, save the images;
in a case where the PCI is to be performed, plan the PCI;
in a case where the PCI is performed, assess or evaluate procedural success of the PCI;
evaluate the physiology of the object or target; and/or
in a case where the object is a vessel or blood vessel, evaluate the physiology of the vessel and/or a lesion of the vessel.

7. The image processing apparatus of claim 1, wherein the one or more processors further operate to reduce a cost of using the image processing apparatus and to reduce an interventional risk during PCI procedure(s) by avoiding wire or other object insertion.

8. The image processing apparatus of claim 1, wherein the one or more processors further operate to one or more of the following:
(i) display an image for each of the one or more imaging modalities on a display, wherein the one or more imaging modalities include one or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view;
(ii) display an image for each of the one or more imaging modalities on a display, wherein the one or more imaging modalities include two or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; and/or
(iii) change or update the displays for each of the one or more imaging modalities based on a calculated FFR and/or based on a request to update or change the displays after calculating the FFR.

9. The image processing apparatus of claim 1, wherein the one or more processors further operate to one or more of the following:
(i) receive information for an interventional device to be used for a Percutaneous Coronary Intervention (PCI); and/or
(ii) in a case where the interventional device is a stent, perform one or more of: detecting stent expansion or underexpansion, detecting stent apposition or malapposition, performing co-registration, performing imaging, displaying a notification regarding the detected stent expansion or underexpansion, displaying a notification regarding the detected stent apposition or malapposition, and confirming stent placement.

10. The image processing apparatus of claim 1, wherein the one or more processors operate to one or more of the following:
(i) employ information on a two-dimensional (2D) and/or three-dimensional (3D) structure or structures for the target or object to create or construct/reconstruct a computational fluid dynamics (CFD) model or result for the target or object;
(ii) use 2D or 3D results and/or 2D or 3D structure(s) and calculate the one or more FFR values and/or one or more instantaneous wave-free ratio (iFR) values;
(iii) employ computational fluid dynamics (CFD) to calculate one or more pressures and to have or obtain the one or more FFR values and/or one or more instantaneous wave-free ratio (iFR) values;
(iv) in a case where it is determined that the stenosis and/or the another medical condition exists in or overlaps with the one or more of the calculated one or more areas of the detected one or more arterial branches based on the calculated one or more FFR values, provide information on one or more treatment options for the stenosis and/or the another medical condition, and/or provide information to determine or assist in determining whether to treat the stenosis and/or the another medical condition;
(v) use the one or more FFR values and/or one or more instantaneous wave-free ratio (iFR) values in real-time;
(vi) calculate pressure(s) and/or include a lamp parameter/circuit analog model;
(vii) include or use an Optical Coherence Tomography (OCT) or Intravascular Ultrasound (IVUS) images or frames FFR method that uses anatomic information; and/or
(viii) process anatomic information where the anatomic information includes at least a volume of a vessel.

11. A method for calculating Fractional Flow Reserve (FFR) values, the method comprising:
obtaining one or more intravascular images of one or more imaging modalities of an object or target during a pullback of a probe or catheter;
detecting one or more arterial branches in the one or more intravascular images;
calculating or determining a pressure loss or change of the one or more arterial branches detected in the one or more intravascular images;
automatically calculating one or more Fractional Flow Reserve (FFR) values using the one or more intravascular images and using the calculated or determined pressure loss or change of the one or more arterial branches;
automatically calculating one or more areas of the detected one or more arterial branches; and
providing or displaying information to determine or assist in determining whether a stenosis and/or another medical condition exists in or overlaps with one or more of the calculated one or more areas of the detected one or more arterial branches based on the calculated one or more FFR values.

12. The method of claim 11, further comprising one or more of the following:
detecting lumen area(s) using a lumen detection method or technique;
detecting a minimum lumen area (As) and defining a stenotic area (L);
constructing a carpet view of the pullback;
in a case where an arterial branch of the detected one or more arterial branches is within or has a portion that passes through or is within, or the calculated area or areas of the arterial branch is within or overlaps with, the stenotic area, reducing a velocity of a fluid or other object passing through the branch or lumen;
calculating a diastolic and systolic Stenotic Flow Reserve(s) (SFR) using the velocity, the stenotic area (L), and the minimum lumen area (As); and/or
using the SFR to calculate the Fractional Flow Reserve (FFR).

13. (canceled)

14. The method of claim 11, further comprising detecting a stenotic area in the one or more intravascular images and calculating the FFR values for the stenotic area only where the pressure loss or change of the one or more arterial branches is occurring.

15. The method of claim 11, wherein the object or target is an organ, a tissue, a sample, a portion of a patient, a vessel, a blood vessel, or a patient.

16. The method of claim 11, further comprising:
determining whether a Percutaneous Coronary Intervention (PCI) is needed for the object or target;
in a case where it is determined that the object or target needs the PCI, performing the PCI, or, in a case where it is determined that the object or target does not need the PCI, saving the images in a memory;
in a case where the PCI is to be performed, planning the PCI;
in a case where the PCI is performed, assessing or evaluating procedural success of the PCI;
evaluating the physiology of the object or target; and/or
in a case where the object is a vessel or blood vessel, evaluating the physiology of the vessel and/or a lesion of the vessel.

17. The method of claim 11, further comprising: reducing a cost of calculating the one or more FFR values as compared to a case not using the method, and reducing an interventional risk during PCI procedure(s).

18. The method of claim 11, further comprising one or more of the following:
(i) displaying an image for each of the one or more imaging modalities on a display, wherein the one or more imaging modalities include one or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view;
(ii) displaying an image for each of the one or more imaging modalities on a display, wherein the one or more imaging modalities include two or more of the following: an imaging modality for a tomography image; an imaging modality for an Optical Coherence Tomography (OCT) image; an imaging modality for a fluorescence image; an imaging modality for a near-infrared fluorescence (NIRF) image; an imaging modality for a near-infrared fluorescence (NIRF) image in a predetermined view (e.g., a carpet view, an indicator view, etc.); an imaging modality for a near-infrared auto-fluorescence (NIRAF) image; an imaging modality for a near-infrared auto-fluorescence (NIRAF) image in a predetermined view (e.g., a carpet view, an indicator view, etc.); an imaging modality for a three-dimensional (3D) rendering; an imaging modality for a 3D rendering of a vessel; an imaging modality for a 3D rendering of a vessel in a half-pipe view or display; an imaging modality for a 3D rendering of the object; an imaging modality for a lumen profile; an imaging modality for a lumen diameter display; an imaging modality for a longitudinal view; computer tomography (CT); Magnetic Resonance Imaging (MRI); Intravascular Ultrasound (IVUS); an imaging modality for an X-ray image or view; and an imaging modality for an angiography view; and/or
(iii) changing or updating the displays for each of the one or more imaging modalities based on a calculated FFR and/or based on a request to update or change the displays after calculating the FFR.

19. The method of claim 11, further comprising one or more of the following:
(i) receiving information for an interventional device to be used for a Percutaneous Coronary Intervention (PCI); and/or
(ii) in a case where the interventional device is a stent, performing one or more of: detecting stent expansion or underexpansion, detecting stent apposition or malapposition, performing co-registration, performing imaging, displaying a notification regarding the detected stent expansion or underexpansion, displaying a notification regarding the detected stent apposition or malapposition, and confirming stent placement.

20. The method of claim 11, further comprising one or more of the following:
(i) employing information on a two-dimensional (2D) and/or three-dimensional (3D) structure or structures for the object to create or construct/reconstruct a computational fluid dynamics (CFD) model or result for the object;
(ii) using 2D or 3D results and/or 2D or 3D structure(s) and calculating the one or more FFR values and/or one or more instantaneous wave-free ratio (iFR) values;
(iii) employing computational fluid dynamics (CFD) to calculate one or more pressures and to have or obtain the one or more FFR values and/or one or more instantaneous wave-free ratio (iFR) values;
(iv) in a case where it is determined that the stenosis and/or the another medical condition exists in or overlaps with the one or more of the calculated one or more areas of the detected one or more arterial branches based on the calculated one or more FFR values, providing information on one or more treatment options for the stenosis and/or the another medical condition, and/or providing information to determine or assist in determining whether to treat the stenosis and/or the another medical condition;
(v) using the one or more FFR values and/or one or more instantaneous wave-free ratio (iFR) values in real-time;
(vi) calculating pressure(s) and/or include a lamp parameter/circuit analog model;
(vii) including or using an Optical Coherence Tomography (OCT) or Intravascular Ultrasound (IVUS) images or frames FFR method that uses anatomic information; and/or
(viii) processing anatomic information where the anatomic information includes at least a volume of a vessel.

21. A non-transitory computer-readable storage medium storing at least one program for causing a computer to execute a method for calculating one or more Fractional Flow Reserve (FFR) values, the method comprising:
obtaining one or more intravascular images of one or more imaging modalities of an object or target during a pullback of a probe or catheter;
detecting one or more arterial branches in the one or more intravascular images;
calculating or determining a pressure loss or change of the one or more arterial branches detected in the one or more intravascular images;
automatically calculating one or more Fractional Flow Reserve (FFR) values using the one or more intravascular images and using the calculated or determined pressure loss or change of the one or more arterial branches;
automatically calculating one or more areas of the detected one or more arterial branches; and
providing or displaying information to determine or assist in determining whether a stenosis and/or another medical condition exists in or overlaps with one or more of the calculated one or more areas of the detected one or more arterial branches based on the calculated one or more FFR values.

22. The storage medium of claim 21, wherein the method further comprises one or more of the following:
detecting lumen area(s) using a lumen detection method or technique;
detecting a minimum lumen area (As) and defining a stenotic area (L);
constructing a carpet view of the pullback;
in a case where an arterial branch of the detected one or more arterial branches is within or has a portion that passes through or is within, or the calculated area or areas of the arterial branch is within or overlaps with, the stenotic area, reducing a velocity of a fluid or other object passing through the branch or lumen;
calculating a diastolic and systolic Stenotic Flow Reserve(s) (SFR) using the velocity, the stenotic area (L), and the minimum lumen area (As); and/or
using the SFR to calculate the Fractional Flow Reserve (FFR).
